(19) United States
(12) Patent Application Publication
(10) Pub. No.: US 2013/0345517 A1
(43) Pub. Date: Dec. 26, 2013

(54) LIGHT SOURCE APPARATUS AND ENDOSCOPE SYSTEM

(71) Applicant: FUJIFILM CORPORATION, Tokyo (JP)

(72) Inventors: Yoshinori MORIMOTO, Ashigarakami-gun (JP); Eiji OHASHI, Ashigarakami-gun (JP); Maki SAITO, Ashigarakami-gun (JP)

(21) Appl. No.: 13/921,504

(22) Filed: Jun. 19, 2013

(30) Foreign Application Priority Data
Jun. 20, 2012 (JP) 2012-138785

(51) Int. Cl. A61B 1/06; H05B 37/02; A61B 1/07; F21V 33/00

(52) U.S. Cl. CPC A61B 1/0661; F21V 33/0068; H05B 37/02; A61B 1/07
USPC 600/178; 362/574; 315/151; 362/554

(57) ABSTRACT

A light source apparatus for supplying an endoscope with light includes at least one light source unit for generating the light. A transparent light homogenizer includes an entrance end face, disposed on a proximal side in an optical axis direction, for receiving entry of the light, an exit end face, disposed on a distal side in the optical axis direction, for exiting the light traveling from the entrance end face, and a reflective interface, formed in a tubular shape between the entrance end face and the exit end face, for total reflection of the light in an internal manner and for directing the light in the optical axis direction. A photo sensor is disposed on the reflective interface, for measuring a light amount of the light. The light homogenizer regularizes the light amount of the light from the exit end face in a radial direction.

### BACKGROUND OF THE INVENTION

1. Field of the Invention

The present invention relates to a light source apparatus and an endoscope system. More particularly, the present invention relates to a light source apparatus and an endoscope system, in which a light amount can be measured only within the light source apparatus in a simplified structure.

2. Description Related to the Prior Art

An endoscope system for diagnosis of a body cavity is widely used in a medical field. The endoscope system includes an endoscope, a light source apparatus, and a processing apparatus. The endoscope includes an elongated tube and a tip device. The elongated tube is entered in the body cavity. The tip device is disposed at a distal end of the elongated tube, images an object in the body cavity, and outputs an image signal. The light source apparatus supplies the endoscope with light for illumination. The processing apparatus processes the image signal output by the endoscope. There are lighting windows and an imaging window formed in the tip device. The lighting windows emit light to the object. The imaging window receives object light from the object for imaging. A light guide device is incorporated in the elongated tube, and has a fiber bundle in which plural optical fibers are bundled. The light guide device guides the light generated by the light source apparatus in the distal direction to the lighting windows in the tip device.

JP-A 2011-183099 discloses examples of a light source in the light source apparatus for emitting white light, such as a xenon lamp and halogen lamp. The white light is condensed by a condenser lens, and supplied to the endoscope in the light source apparatus. The light source apparatus in JP-A 2011-183099 includes a color separation filter and a photo sensor for light measurement. The color separation filter separates white light from the light source into B, G and R components. The photo sensor measures light amounts of the B, G and R components of light. The light amounts from the photo sensor is used for output adjustment (calibration) and gain adjustment of the image signal in the processing apparatus for the purpose of correcting a color balance of the image.

In the light source apparatus of JP-A 2011-183099, it is unnecessary to use the endoscope the photo sensor for light measurement because the photo sensor is built-in. Output adjustment is carried out only in the light source apparatus without its connection with the endoscope.

The light source apparatus of JP-A 2011-183099 includes a beam splitter disposed within an optical path from the light source to the condenser lens. Part of light emitted by the light source is guided by the beam splitter to the photo sensor for light measurement. Also, a disclosed variant of the structure is disposition of the photo sensor within the optical path without using the beam splitter for measuring the light amount.

Also, JP-A 2011-041758 discloses a type of the light source apparatus in which a light emitting device of semiconductor is used as the light source in place of a xenon lamp or halogen lamp, for example, a laser diode (LD) and light-emitting diode (LED).

The light source apparatus includes a plurality of light emitting devices and a rod lens. The light emitting devices emit light of wavelengths different from one another. The rod lens mixes light generated by the light emitting devices, and supplies the endoscope with the light.

However, the beam splitter of JP-A 2011-183099 is a component only for guiding the light to the photo sensor for light measurement. Adding a specialized component such as the beam splitter to the photo sensor causes a problem of increasing the total number of the parts, increasing the manufacturing cost, and enlarging a space of the disposition of the part.

In JP-A 2011-183099, there is a suggestion of disposing the photo sensor for light measurement in the optical path without using the beam splitter. However, a problem arises in that vignetting may occur in an image, because the photo sensor in the optical path may block object light. No solution of such problems is disclosed in JP-A 2011-183099 or JP-A 2011-041758.

### SUMMARY OF THE INVENTION

In view of the foregoing problems, an object of the present invention is to provide a light source apparatus and an endoscope system, in which a light amount can be measured only within the light source apparatus in a simplified structure.

In order to achieve the above and other objects and advantages of this invention, a light source apparatus for supplying an endoscope with light includes at least one light source unit for generating the light. A transparent light guide rod includes an entrance end face, disposed on a proximal side in an optical axis direction, for receiving entry of the light, an exit end face, disposed on a distal side in the optical axis direction, for exiting the light traveling from the entrance end face, and a reflective interface, formed between the entrance end face and the exit end face, for total reflection of the light in an internal manner and for directing the light in the optical axis direction. At least one photo sensor is disposed on the reflective interface, for measuring a light amount of the light.

The light guide rod includes a light homogenizer for regularizing the light amount of the light from the exit end face in a radial direction.

Furthermore, an adhesive agent has a higher refractive index than the light guide rod, for attaching the photo sensor to the reflective interface.

The at least one light source unit is plural light source units. Furthermore, an integrating device is disposed upstream of the light guide rod, for aligning optical paths of the light from the plural light source units together and directing the light to the light guide rod.

Furthermore, a driver drives the light source units by supplying power. A lighting control unit is connected with the driver, operated if a change occurs in the light amount measured by the photo sensor, for canceling the change by adjusting the power.

The plural light source units include first and second light source units between which wavelengths of the light are different from one another.

The at least one photo sensor is a plurality of photo sensors.

At least one of the first and second light source units generates special light for special light imaging.

At least one of the first and second light source units includes a light emitting device of semiconductor.

The light emitting device is a laser diode.

The first light source unit includes a light emitting device of semiconductor for generating first light. Phosphor generates fluorescence by excitation with the first light, to emit mixed light by addition of the fluorescence to the first light.

The at least one photo sensor includes a first photo sensor sensitive to a first wavelength band of the first light. A second photo sensor is sensitive to a second wavelength band being different from the first wavelength band and containing a wavelength of the fluorescence.

The integrating device includes plural input fiber ends, each of which is constituted by a fiber bundle of plural optical fibers, for receiving the light from one of the plural light source units. An output fiber end emits the light to the light guide rod. A routing section collects the optical fibers from the plural input fiber ends into one fiber bundle in the optical axis direction, to constitute the output fiber end.

In another preferred embodiment, furthermore, an optical coupler has the light guide rod, and the integrating device formed to extend upstream of the light guide rod.

Furthermore, a receptacle connector contains at least a portion of the light guide rod on a side of the exit end face, and for connection with a proximal connector of the endoscope. A monitoring unit monitors reflected light on the reflective interface with the photo sensor upon application of the light from the light source unit through the light guide rod, and checks whether connection between the receptacle connector and the proximal connector is appropriate by evaluating the reflected light.

Also, an endoscope system including an endoscope and a light source apparatus for supplying the endoscope with light is provided. The light source apparatus includes at least one light source unit for generating the light. A transparent light guide rod includes an entrance end face, disposed on a proximal side in an optical axis direction, for receiving entry of the light, an exit end face, disposed on a distal side in the optical axis direction, for exiting the light from the entrance end face, and a reflective interface, formed between the entrance end face and the exit end face, for total reflection of the light in an internal manner and for directing the light in the optical axis direction. At least one photo sensor is disposed on the reflective interface, for measuring a light amount of the light.

Accordingly, alight amount can be measured only within the light source apparatus in a simplified structure, because of the photo sensor disposed suitably on the tubular shape.

### BRIEF DESCRIPTION OF THE DRAWINGS

The above objects and advantages of the present invention will become more apparent from the following detailed description when read in connection with the accompanying drawings, in which:

FIG. 1 is a perspective illustrating an endoscope system;

FIG. 2 is a front elevation illustrating a tip device of an endoscope;

FIG. 3 is a block diagram schematically illustrating the endoscope system;

FIG. 4 is a graph illustrating spectral distribution of light;

FIG. 5 is a graph illustrating an absorption spectrum of hemoglobin;

FIG. 6 is a graph illustrating a scattering coefficient of tissue;

FIG. 7 is a graph illustrating spectral distribution of a color micro filter;

FIG. 8A is a timing chart illustrating irradiation and imaging with the light;

FIG. 8B is a timing chart illustrating irradiation and imaging in a vessel enhancement mode;

FIG. 8C is a timing chart illustrating irradiation and imaging in an oxygen saturation monitoring mode;

FIG. 9A is a flow chart illustrating image processing in a normal imaging mode;

FIG. 9B is a flow chart illustrating image processing in the vessel enhancement mode;

FIG. 9C is a flow chart illustrating image processing in the oxygen saturation monitoring mode;

FIG. 10 is a perspective illustrating an optical routing device and a light source unit;

FIG. 11 is an explanatory view in a section illustrating arrangement of optical fibers;

FIG. 12 is an explanatory view in a section illustrating a photo sensor for light measurement and an output adjusting device;

FIG. 13 is an explanatory view in a section illustrating mounting of the photo sensor;

FIG. 14 is a table illustrating information in an LUT;

FIG. 15 is a perspective illustrating a first light source unit;

FIG. 16 is an explanatory view in a section illustrating a divergence angle corrector of the first light source unit;

FIG. 17 is a perspective illustrating a second light source unit;

FIG. 18 is an explanatory view in a section illustrating a divergence angle corrector of the second light source unit;

FIG. 19 is a flow chart illustrating output adjustment (calibration);

FIG. 20 is a perspective illustrating another preferred light source apparatus with a plurality of photo sensors for light measurement;

FIG. 21 is an explanatory view in a section illustrating a photo sensor for light measurement and an output adjusting device;

FIG. 22 is a graph illustrating a spectral sensitivity of a photo sensor S2;

FIG. 23 is a graph illustrating a spectral sensitivity of a photo sensor S3;

FIG. 24 is a flow chart illustrating output adjustment;

FIG. 25 is an explanatory view in a section illustrating still another preferred light source apparatus with a monitoring unit and a presence sensor;

FIG. 26 is an explanatory view in a section illustrating an appropriate mounted condition of a connector;

FIG. 27 is an explanatory view in a section illustrating an improper mounted condition;

FIG. 28 is a flow chart illustrating a function of testing the mounted condition;

FIG. 29 is a explanatory view in a section illustrating another preferred light homogenizer and an optical routing device formed together with the light homogenizer.

### DETAILED DESCRIPTION OF THE PREFERRED EMBODIMENT(S) OF THE PRESENT INVENTION

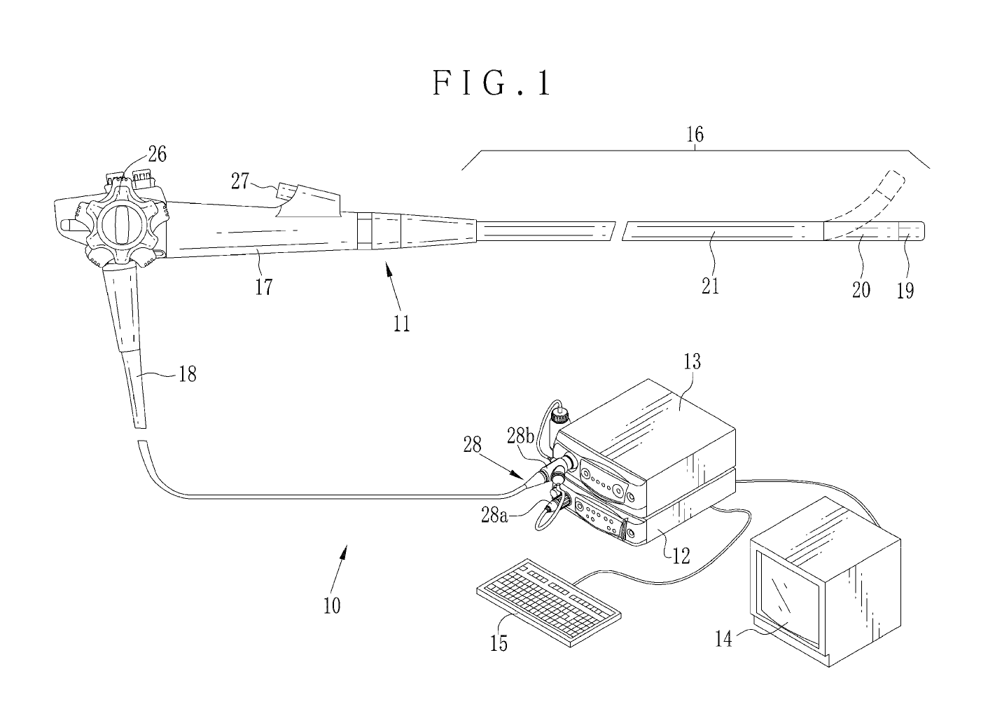

In FIG. 1, an endoscope system 10 includes an endoscope 11, a processing apparatus 12, a light source apparatus 13 and a monitor display panel 14. The endoscope 11 images an object in a body cavity, and generates an image signal. The processing apparatus 12 creates an object image according to the image signal. The light source apparatus 13 supplies the endoscope 11 with light for illuminating the object. The display panel 14 displays the image. A console unit 15 is a user interface of the processing apparatus 12 and includes various elements such as a keyboard, mouse and the like.

The endoscope system 10 is operable in a normal imaging mode (color imaging mode) and a special imaging mode. In the normal imaging mode, an object of interest is imaged with white light. In the special imaging mode, special light is used to image blood vessels in a region of interest. In the special imaging mode, a pattern of the blood vessels or an oxygen saturation level is recognized to diagnose a tumor between benign and malignant conditions. An example of the special light is narrow band light with a high wavelength band because of high absorbance in blood hemoglobin. Examples of the special imaging mode include a vessel enhancement mode and an oxygen saturation monitoring mode. In the vessel enhancement mode, a vessel-enhanced image in which the vessels are enhanced is output and displayed. In the oxygen saturation monitoring mode, a special image in which the oxygen saturation level (S02 level) of the blood hemoglobin is output and displayed.

The endoscope 11 includes an elongated tube 16, a handle 17 and a universal cable 18. The elongated tube 16 is entered in a gastrointestinal tract of a body. The handle 17 is disposed on a proximal end of the elongated tube 16. The universal cable 18 extends between the handle 17 and the processing apparatus 12 and the light source apparatus 13 for connection.

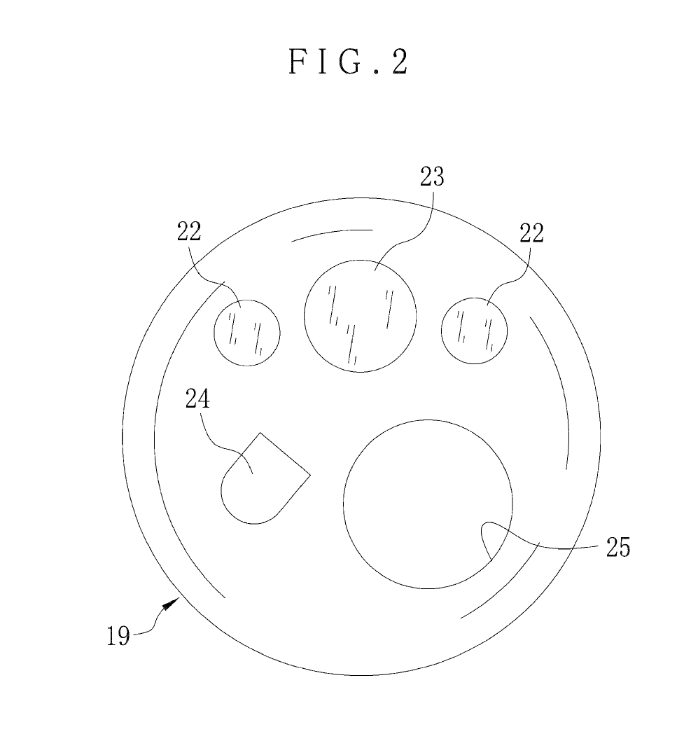

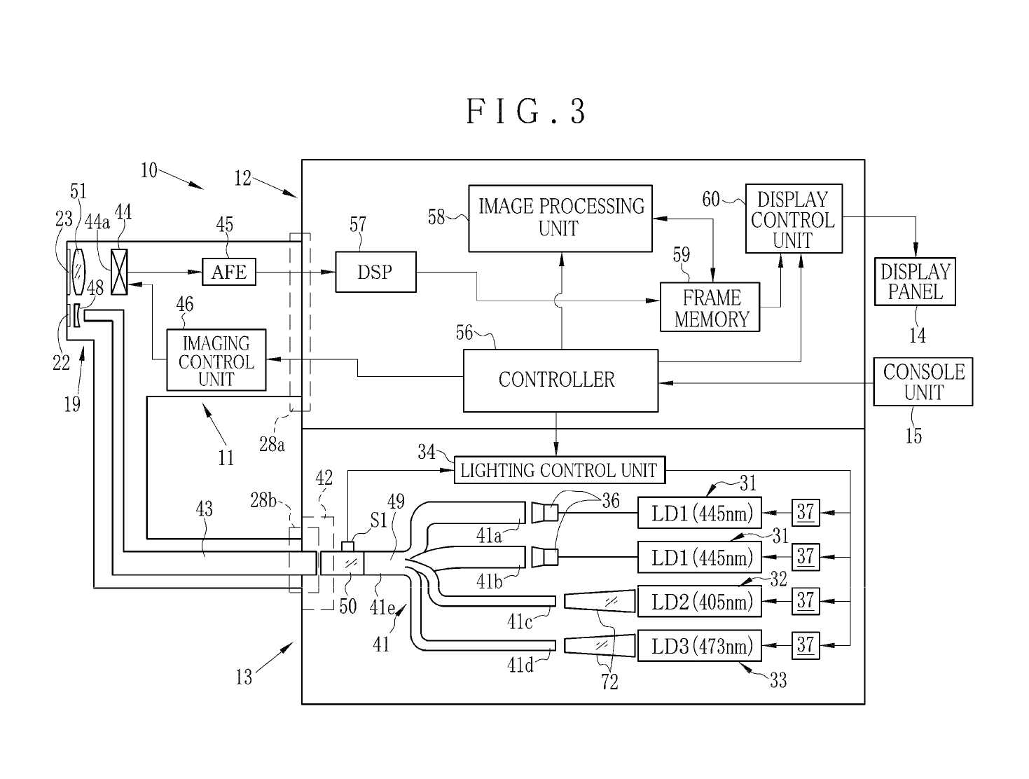

The elongated tube 16 includes a tip device 19, a steering device 20 and a flexible tube device 21 arranged in a proximal direction. In FIG. 2, a distal surface of the tip device 19 has lighting windows 22, an imaging window 23, a nozzle spout of a fluid supply nozzle 24 for air and water, and a distal instrument opening 25. The lighting windows 22 apply light to an object in a body cavity. The imaging window 23 receives object light reflected by the object. The fluid supply nozzle 24 ejects air and/or water for cleaning the imaging window 23. The distal instrument opening 25 causes a medical instrument to protrude, such as a forceps, electrocautery device and the like. An imaging unit 44 is disposed behind the imaging window 23 together with a lens system for image forming. See FIG. 3.

The steering device 20 includes a plurality of link elements connected serially with one another. Steering wheels 26 are mounted on the handle 17, and rotated to steer the steering device 20 up and down and to the right and left. The tip device 19 is directed by bending the steering device 20 as desired by a doctor or operator. The flexible tube device 21 is so flexible as to enter a body cavity in a tortuous form smoothly, such as an esophagus, intestines and the like. The elongated tube 16 contains a communication line and a light guide device 43 of FIG. 3. The communication line transmits a drive signal for driving the imaging unit 44, and an image signal output by the imaging unit 44. The light guide device 43 directs light supplied by the light source apparatus 13 to the lighting windows 22.

A proximal instrument opening 27 is formed in the handle 17 for entry of the medical instrument. Also, the handle 17 has the steering wheels 26, a fluid supply button, a recording button and the like. The fluid supply button is depressible for supply of water and/or air. The recording button is depressible for recording a still image.

The universal cable 18 has the communication line, the light guide device 43 and the like extending from the elongated tube 16. A proximal connector 28 is mounted on a proximal end of the universal cable 18 on the side of the processing apparatus 12 and the light source apparatus 13. The proximal connector 28 is in a composite form and includes a first connection plug 28a for communication and a second connection plug 28b for lighting. An end portion of the communication line is contained in the first connection plug 28a, which is connectively coupled with the processing apparatus 12. An entrance end portion of the light guide device 43 is contained in the second connection plug 28b, which is connectively coupled with the light source apparatus 13.

In FIG. 3, the light source apparatus 13 includes first light source units 31 or modules, a second light source unit 32 or module, a third light source unit 33 or module, and a lighting control unit 34. The light source units 31-33 emit light of wavelengths different between those. The lighting control unit 34 controls the light source units 31-33. The lighting control unit 34 controls a sequence of driving and synchronization of the elements in the light source apparatus 13.

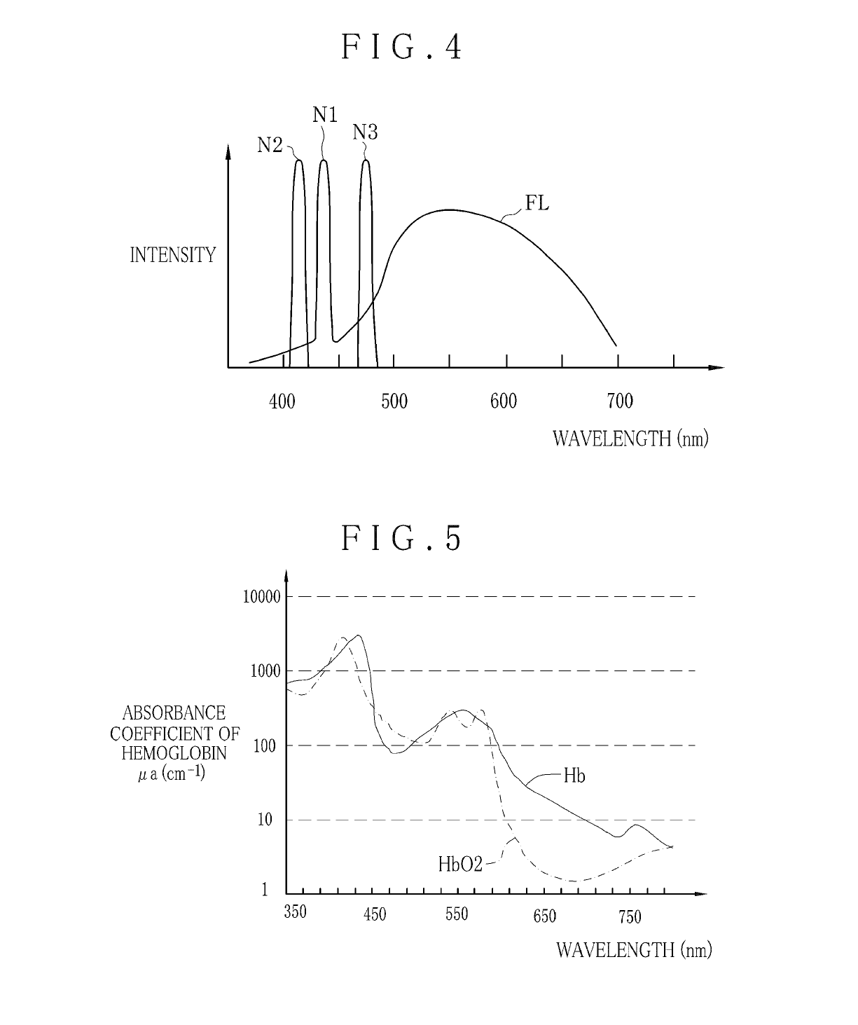

The light source units 31-33 have laser diodes LD1-LD3 for emitting narrow band light of a predetermined wavelength. In FIG. 4, the laser diode LD1 emits narrow band light N1 with a center wavelength of 445 nm in a limited band of 440 plus or minus 10 nm in the blue range. The laser diode LD2 emits narrow band light N2 with a center wavelength of 405 nm in a limited band of 410 plus or minus 10 nm in the blue range. The laser diode LD3 emits narrow band light N3 with a center wavelength of 473 nm in a limited band of 470 plus or minus 10 nm in the blue range. Available examples of the laser diodes LD1-LD3 are an InGaN type, InGaNAs type, GaNAs type and the like. A preferable type of the laser diodes LD1-LD3 is a broad area type of which a stripe width (width of waveguide) is large for a structure of high output.

The first light source units 31 emit white light for normal imaging. Phosphor 36 is provided in the first light source units 31 in combination with the laser diode LD1. In FIG. 4, the phosphor 36 is excited by the narrow band light N1 of 445 nm in a blue range emitted by the laser diode LD1, and emits fluorescence FL in a wavelength band from green to red. The phosphor 36 partially absorbs the narrow band light N1 to emit the fluorescence FL, and causes the remainder of the narrow band light N1 to pass. The transmitted component of the narrow band light N1 is diffused by the phosphor 36. The transmitted component is mixed with the fluorescence FL to obtain white light. Examples of the phosphor 36 are a YAG type, BAM type (BgMgAl₁₀O₁₇ type) and the like. The number of the first light source units 31 is two for acquiring a large light amount of white light.

The second light source unit 32 illuminates for the purpose of blood vessel enhancement. In FIG. 5, an absorption spectrum of blood hemoglobin is illustrated. An absorbance coefficient μa in blood has dependency to the wavelength, rises abruptly in a wavelength band under 450 nm, and comes to a first peak at a wavelength near to 405 nm. Also, the absorbance coefficient comes to a second peak at a wavelength of 530-560 nm being lower than the first peak. When light of a wavelength with a high absorbance coefficient μa is applied to an object of interest, an image with clearly high contrast between vessels and other tissue can be formed because of characteristically high absorption in the vessels.

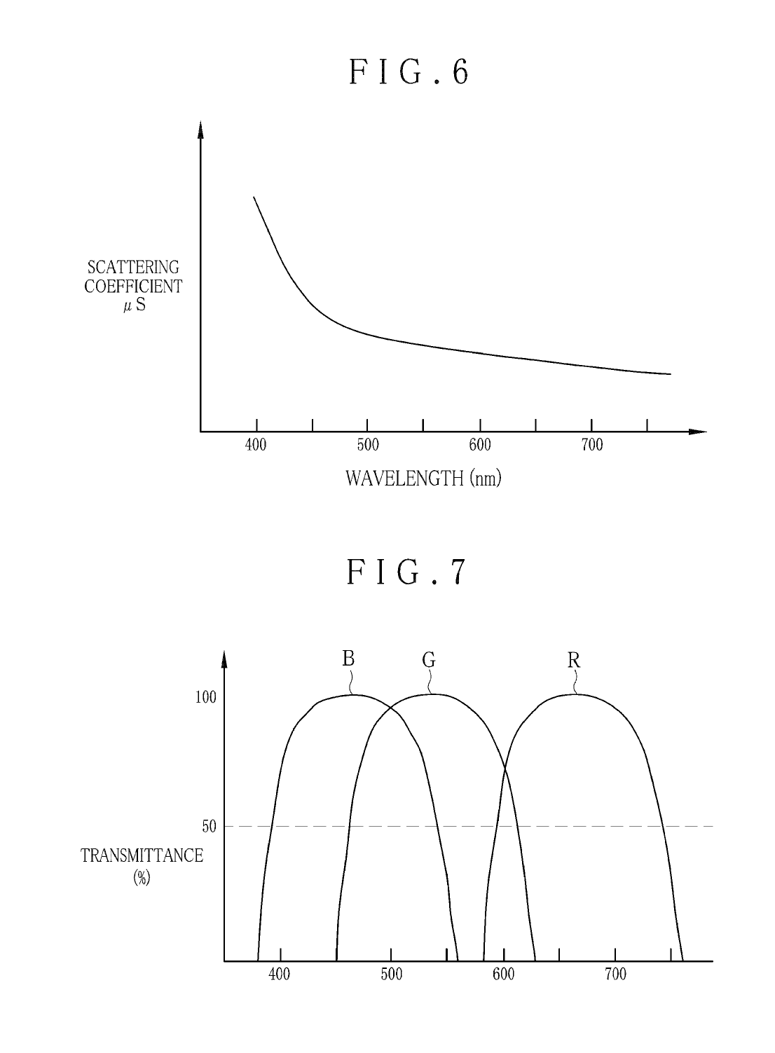

In FIG. 6, a scattering property of tissue for light has dependency to a wavelength. A scattering coefficient μs increases according to shortness of the wavelength. The scattering influences to a penetration depth of light into the tissue of a body. According to highness of the scattering, light reflected near to the surface of the mucosa of the tissue increases, and light reaching the moderately deep or deep layer decreases. Thus, the penetration depth decreases according to the decrease of the wavelength, and increases according to the increase of the wavelength. Wavelengths of light for vessel enhancement are selected in consideration of absorption of hemoglobin, and the scattering property of tissue for light.

The narrow band light N2 of 405 nm emitted by the second light source unit 32 has a small penetration depth, and used for emphasizing surface vessels because of high absorption in the surface vessels. The surface vessels can be imaged with high contrast in a display image by use of the narrow band light N2. Also, a green component in white light emitted by the first light source units 31 is used for emphasizing deep vessels and moderately deep vessels. In the absorption spectrum of FIG. 5, the absorbance coefficient changes more gradually in the green region of 530-560 nm than in the blue region of 450 nm or lower. A band of the light for deep vessels and moderately deep vessels does not need to be so narrow as a band of the blue light. As will be described later, a green component separated from the white color by a micro color filter of green in the imaging unit 44 is used.

The third light source unit 33 is for oxygen saturation monitoring. In FIG. 5, an absorption spectrum Hb is according to the deoxyhemoglobin without bonding to oxygen. An absorption spectrum HbO2 is according to the oxyhemoglobin bonded to oxygen. The deoxyhemoglobin and oxyhemoglobin have absorption properties different from one another. There occurs a difference in the absorbance coefficient μa except for an isosbestic point (intersection point between the spectra Hb and HbO2) of an equal absorbance coefficient μa. Reflectivity is changed upon a change of the oxygen saturation level even in application of light of an equal intensity and equal wavelength, because of the difference in the absorbance coefficient μa. In the oxygen saturation monitoring mode, the narrow band light N3 emitted by the third light source unit 33 of a wavelength 473 nm with a difference in the absorbance coefficient μa is used for measuring the oxygen saturation level.

Drivers 37 are controlled by the lighting control unit 34 to turn on and off the laser diodes LD1-LD3 and control their light amounts. To this end, the lighting control unit 34 generates drive pulses to drive the laser diodes LD1-LD3. A duty factor of the drive pulses is controlled according to the PWM (pulse width modulation) control to change a drive current. Note that the control of the drive current (or power) for the laser diodes LD1-LD3 can be the PAM (pulse amplitude modulation) control to change the amplitude instead of the PWM control.

The lighting control unit 34 carries out output adjustment (calibration) of the light source units 31-33 as described later. In the laser diodes LD1-LD3, the drive current for obtaining a predetermined light amount is changed according to degradation with time or environmental condition. A photo sensor S1 for light measurement is provided in the light source apparatus 13 for measuring light amounts of the light source units 31-33. The lighting control unit 34 adjusts the drive current of the laser diodes LD1-LD3 according to a light amount signal output by the photo sensor S1.

An optical routing device 41 (integrating device) for guiding light is disposed downstream of the light source units 31-33. The optical routing device 41 aligns optical paths from the light source units 31-33 in one direction. As an entrance end of the light guide device 43 of the endoscope 11 is single, the optical paths from the light source units 31-33 are aligned by the optical routing device 41 for supply of light from those to the endoscope 11. The optical routing device 41 has four input fiber ends 41a, 41b, 41c and 41d (branch waveguides), a routing section 49, and an output fiber end 41e (end waveguide).

The first light source units 31 are opposed to the input fiber ends 41a and 41b of the optical routing device 41. The second and third light source units 32 and 33 are opposed to respectively the input fiber ends 41c and 41d of the optical routing device 41.

A receptacle connector 42 or mating coupler is disposed for connection with the second connection plug 28b of the endoscope 11. The output fiber end 41e of the optical routing device 41 is positioned near to the receptacle connector 42. A light homogenizer 50 is disposed downstream of the output fiber end 41e. Light from the light source units 31-33 upon entry in the optical routing device 41 is passed through the light homogenizer 50 and supplied to the light guide device 43 of the endoscope 11 disposed in the second connection plug 28b.

The endoscope 11 includes an analog processing unit 45 (AFE) and an imaging control unit 46 in addition to the light guide device 43 and the imaging unit 44. The light guide device 43 is a fiber bundle including plural optical fibers bundled together. When the proximal connector 28 is connectively coupled to the light source apparatus 13, an entrance end face of the light guide device 43 is opposed to an exit end face of the light homogenizer 50. The exit end face of the light guide device 43 is branched in two portions upstream of the lighting windows 22, for guiding light to both of the lighting windows 22.

An illumination lens 48 is disposed behind the lighting windows 22. Light generated by the light source apparatus 13 is guided by the light guide device 43 to the illumination lens 48, and emitted by the lighting windows 22 toward a region of interest. An example of the illumination lens 48 is a concave lens which increases a divergence angle of the light output by the light guide device 43. It is possible to apply the light to the region of interest in an enlarged manner.

An objective lens system 51 and the imaging unit 44 are disposed behind the imaging window 23. Object light reflected by the object becomes incident upon the lens system 51 through the imaging window 23. An imaging surface 44a is disposed on the imaging unit 44, where the light is focused by the lens system 51.

The imaging unit 44 is a CCD or CMOS image sensor. The imaging surface 44a has plural photoelectric elements such as photo diodes arranged in plural arrays. The imaging unit 44 converts light received by the imaging surface 44a photoelectrically, and stores signal charge according to a light amount of received light at respective pixels. The signal charge is converted into a voltage signal by an amplifier, and is read. The voltage signal is an image signal, which is output by the imaging unit 44 to the analog processing unit 45.

The imaging unit 44 is a full-color type. Micro color filters of three colors of B, G and R are disposed on the imaging surface 44a, and assigned to respectively pixels. In FIG. 7, spectral distribution of the micro color filters is illustrated. White light emitted by the first light source units 31 is separated by the micro color filters into B, G and R light components. An example of arrangement of the micro color filters is Bayer arrangement.

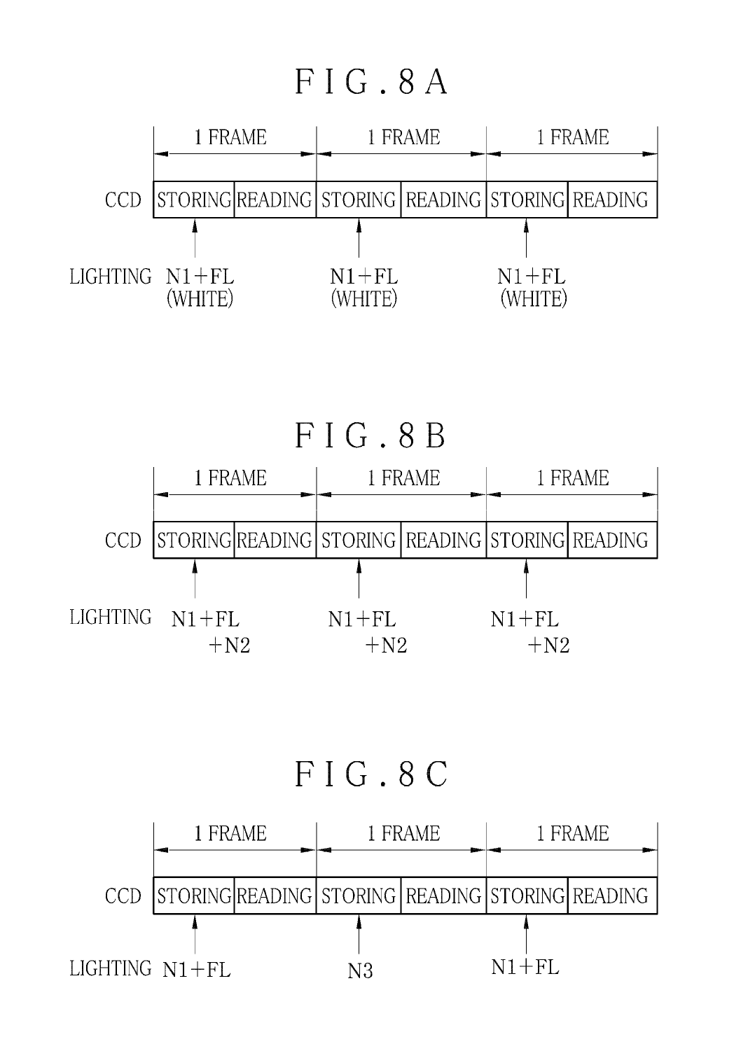

In FIGS. 8A, 8B and 8C, the imaging unit 44 in the normal imaging mode carries out storing and reading, and stores signal charge within a period of acquisition of one frame in the storing, and reads the stored signal charge in the reading. In FIG. 8A, the laser diode LD1 is turned on in the normal imaging mode according to the sequence of the storing. White light obtained from the narrow band light N1 and the fluorescence FL is applied to an object of interest. Reflected light from the object is received by the imaging unit 44. In the imaging unit 44, white color is separated in the color separation by a micro color filter. Reflected light corresponding to the narrow band light N1 is received by the B pixels. A G component in the fluorescence FL is received by the G pixels. An R component in the fluorescence FL is received by the R pixels. The imaging unit 44 sequentially outputs image signals B, G and R of one frame according to a frame rate with pixel values of pixels of B, G and R according to the sequence of the reading. The operation of the imaging is repeated in the course of the normal imaging mode.

In the vessel enhancement mode, the second light source unit 32 in addition to the first light source units 31 is turned on according to the sequence of the storing, as illustrated in FIG. 8B. When the first light source units 31 are turned on, the white light (N1+FL) in combination of the narrow band light N1 and fluorescence FL is applied to an object of interest, in a manner similar to the normal imaging mode. When the second light source unit 32 is turned on, the narrow band light N2 and the white light (N1+FL) are applied to the object of interest.

The light after addition of the white light and the narrow band light N2 is separated by the B, G and R micro color filters in the imaging unit 44 in a manner similar to the normal imaging mode. The B pixels in the imaging unit 44 receive the narrow band light N2 in addition to the narrow band light N1. The G pixels receive a G component in the fluorescence FL. The R pixels receive an R component in the fluorescence FL. In the vessel enhancement mode, the imaging unit 44 sequentially outputs the image signals B, G and R according to the frame rate in the sequence of the reading. Those steps of the imaging are repeated in the course of the vessel enhancement mode.

In the oxygen saturation monitoring mode, the first light source units 31 are turned on according to the sequence of the storing as illustrated in FIG. 8C. In response, the white light (N1+FL) is applied to an object of interest in a manner similar to the normal imaging mode. In a second frame, the first light source units 31 are turned off. The third light source unit 33 is turned on to apply narrow band light N3 to the object of interest. Also in the oxygen saturation monitoring mode, the imaging unit 44 outputs image signals B, G and R according to the frame rate in the sequence of the reading.

In the oxygen saturation monitoring mode, the white light (N1+FL) and the narrow band light N3 are used alternately for emission in a manner different from the normal imaging mode and the vessel enhancement mode. Image signals B, G and R corresponding to the white light are output at a first frame. Image signals B, G and R corresponding to the narrow band light N3 are output at a second frame. Information according to the image signals B, G and R changes for each of the frames in correspondence with the illumination light. This sequence of imaging is repeated in the vessel enhancement mode.

In FIG. 3, the analog processing unit 45 includes a correlated double sampling circuit (CDS), an automatic gain control device (AGC) and an A/D converter (all not shown). The correlated double sampling circuit processes an image signal of an analog form from the imaging unit 44 in a correlated double sampling, and eliminates electric noise due to resetting a signal charge. The automatic gain control device amplifies the image signal after eliminating the noise in the correlated double sampling circuit. The A/D converter converts the amplified image signal from the automatic gain control device into a digital image signal of gradation steps according to a predetermined number of bits. The digital image signal is input to the processing apparatus 12.

A controller 56 is incorporated in the processing apparatus 12. The imaging control unit 46 is connected with the controller 56, is synchronized with a base clock signal from the controller 56, and outputs a drive signal to the imaging unit 44. The imaging unit 44 outputs an image signal to the analog processing unit 45 at a predetermined frame rate according to the drive signal from the imaging control unit 46.

The processing apparatus 12 includes a digital signal processor 57 (DSP), an image processing unit 58, a frame memory 59 and a display control unit 60 in addition to the controller 56. The controller 56 has a ROM, RAM and the like. The ROM stores a control program and various data required for the control. The RAM is a working memory for loading the control program. The CPU runs the control program to control various elements of the processing apparatus 12.

The digital signal processor 57 receives the image signal from the imaging unit 44. The digital signal processor 57 separates the image signal into image signals B, G and R, and processes those in the pixel interpolation. Also, the digital signal processor 57 processes the image signals B, G and R in signal processing, for example, white balance correction.

The digital signal processor 57 determines an exposure amount according to the image signals B, G and R, and causes the controller 56 to send an exposure control signal to the light source apparatus 13 so as to increase a light amount if brightness of the image is too low and to decrease the light amount if the brightness of the image is too high. The light source apparatus 13 controls the light amounts of the light source units 31-33 according to the exposure control signal.

The frame memory 59 stores the image data output by the digital signal processor 57 and the processed data processed by the image processing unit 58. The display control unit 60 reads the processed image data from the frame memory 59, converts this into a video signal such as a composite signal and component signal, and outputs the video signal to the display panel 14.

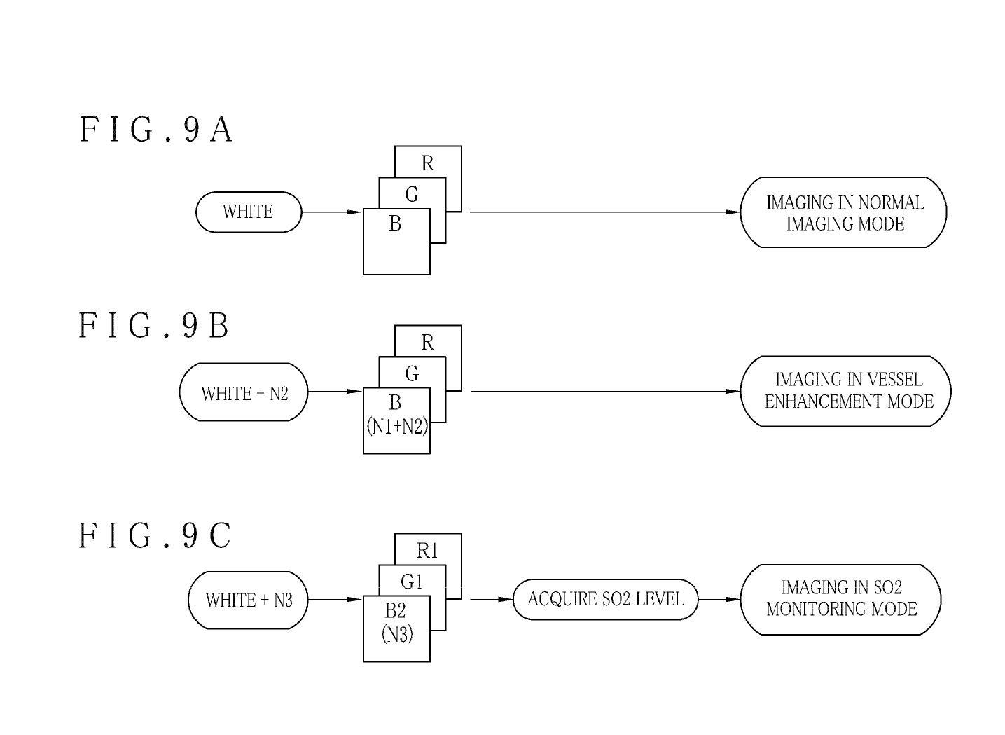

In FIG. 9A, the image processing unit 58 in the normal imaging mode creates an image for the normal imaging according to the image signals B, G and R after color separation in the digital signal processor 57. The image is displayed on the display panel 14. The image processing unit 58 updates the image at each time that the image signals B, G and R in the frame memory 59 are updated.

In FIG. 9B, the image processing unit 58 in the vessel enhancement mode creates an image for the vessel enhancement according to the image signals B, G and R. The image signal B in the vessel enhancement mode has information of a B component in the white light (narrow band light N1 and part of fluorescence FL) and information of the narrow band light N2. Thus, surface vessels are imaged at high contrast. It is known that there is a characteristic pattern of vessels in a tumor or other lesions, for example, density of surface vessels is higher in lesions than in normal tissue. Accordingly, it is preferable to image the surface vessels clearly in the vessel enhancement mode for the purpose of diagnosing benign and malignant conditions of a tumor and the like.

To enhance the surface vessels, areas of the surface vessels are detected according to the image signal B, and are processed for image processing such as edge enhancement. The processed image signal B is combined with a full-color image created from the image signals B, G and R. As a result, the surface vessels are reliably enhanced. It is possible to process areas of images of moderately deep vessels and deep vessels similarly in the vessel enhancement. To this end, the areas of the moderately deep vessels and deep vessels are extracted from the image signal G in which information of those is remarkably contained. The extracted areas are processed for the edge enhancement. The image signal G after the edge enhancement is combined with the full-color image generated from the image signals B, G and R.

The vessel-enhanced image of an object of interest is a full-color image similar to a regular image because of the B, G and R image signals. However, blue density of the image signal B in the vessel enhancement mode is higher than that of the image signal B in the normal imaging mode. It is possible in the vessel enhancement to correct the vessel-enhanced image with color balance near to that of the regular image for the normal imaging mode. The image processing unit 58 generates the vessel-enhanced image at each time that the B, G and R image signals in the frame memory 59 are updated.

Other methods of creating a display image in the vessel enhancement can be used. For example, the object of interest may be displayed in pseudo color representation. An image is created only from the image signals B and G without use of the image signal R, to assign the image signal B to the B and G channels, and assign a signal associated with the image signal G to the R channel of the display panel 14.

In FIG. 9C, the image processing unit 58 in the oxygen saturation monitoring mode processes the image signals G1 and R1 acquired by use of white light and the image signal B2 acquired by use of the narrow band light N3, for obtaining an oxygen saturation level. The pixel value of the image signal B2 includes information of a blood amount or density in addition to the oxygen saturation level. For higher precision, it is necessary to separate information of the blood amount from the pixel value of the pixel signal B2. The image processing unit 58 arithmetically operates between the image signals B and R with high correlation to the blood amount, and separates information of the blood amount from the oxygen saturation level.

Specifically, the image processing unit 58 refers to pixel values of the image signals B2, G1 and R1 at the same points, and obtains a ratio B/G of the pixel value of the image signal B2 to the pixel value of the image signal G1, and a ratio RIG of the pixel value of the image signal R1 to the pixel value of the image signal G1. The image signal G1 is used as a reference signal of a brightness level of an object of interest for normalizing the pixel values of the image signals B2 and R1. Then the oxygen saturation level after removing information of the blood amount is determined according to an initially prepared table of a correlation between the ratios B/G and RIG, the oxygen saturation level and the blood amount. A full-color image according to the image signals B2, G1 and R1 is processed in the color conversion according to the determined value of the oxygen saturation level, so that a display image for the oxygen saturation monitoring mode is created.

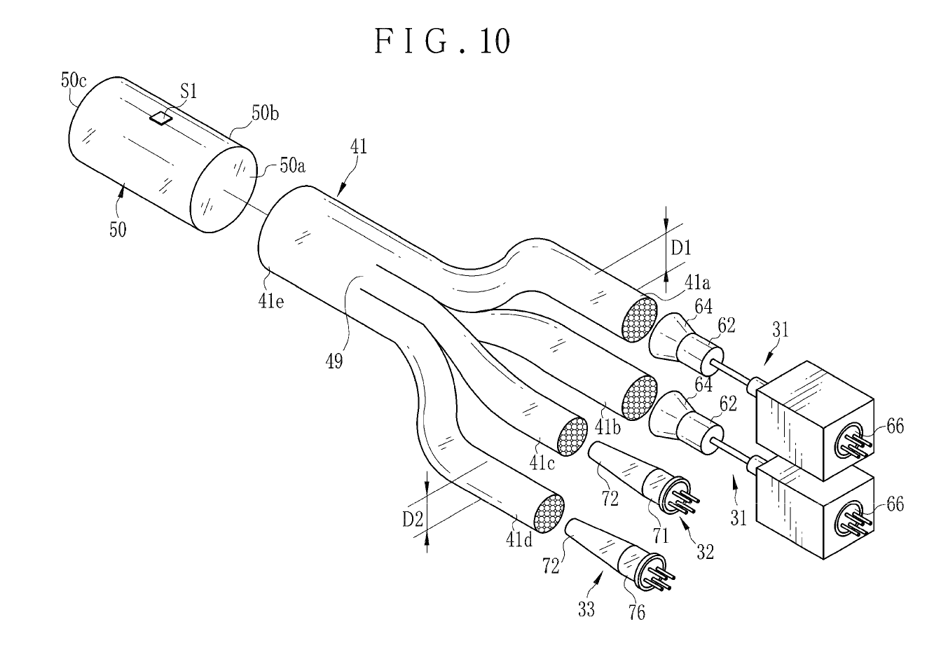

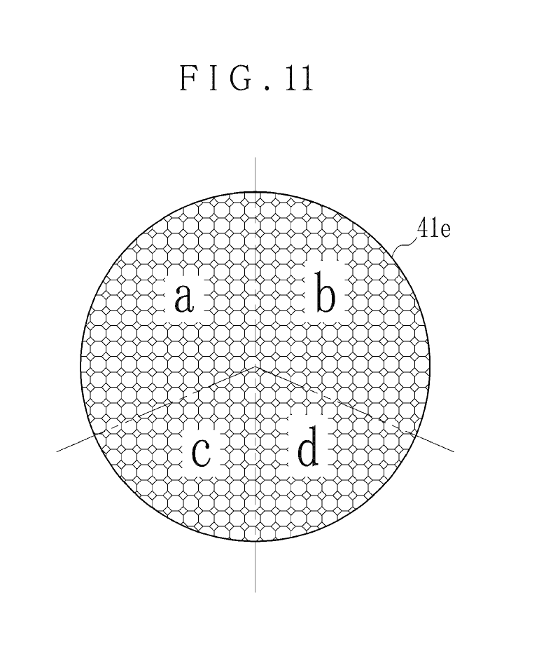

In FIG. 10, the optical routing device 41 in the light source apparatus 13 is a fiber bundle obtained by bundling plural optical fibers in a manner similar to the light guide device 43 of the endoscope 11. All of the optical fibers are collected at the output fiber end 41e of the optical routing device 41, but are split into four groups at the intermediate routing section 49. The input fiber ends 41a-41d are formed by bundling the optical fibers of each of the groups.

A diameter D1 of the input fiber ends 41a and 41b is set different from a diameter D2 of the input fiber ends 41c and 41d by changing the number of optical fibers bundled respectively. The diameter D1 is larger than the diameter D2. This is because the first light source units 31 have the phosphor 36 in contrast with the second and third light source units 32 and 33 without the phosphor 36. A beam diameter of the light flux of the first light source units 31 associated with the input fiber ends 41a and 41b is larger than that of the second and third light source units 32 and 33 associated with the input fiber ends 41c and 41d. Also, one more reason for the difference is that the first light source units 31 emitting white light for the normal imaging should be constructed for a higher light amount than the second and third light source units 32 and 33 for special light imaging.

Specifically, a diameter of the light guide device 43 of the endoscope 11 is approximately 2 mm. A diameter of the output fiber end 41e of the optical routing device 41 is also approximately 2 mm. The diameter D1 of the input fiber ends 41a and 41b is approximately 1.0-1.4 mm. The diameter D2 of the input fiber ends 41c and 41d is approximately 0.5-0.8 mm.

The light homogenizer 50 is disposed at the output fiber end 41e of the optical routing device 41. The light homogenizer 50 regularizes distribution of light of the plural colors from the light source units 31-33 with uniformity.

The light homogenizer 50 is a light guide rod of a cylindrical shape in the optical axis direction, and formed from quartz glass or other transparent material. The light homogenizer 50 includes an entrance end face 50a, an exit end face 50c, and a reflective interface 50b or peripheral surface. The entrance end face 50a receives light exited from the optical routing device 41. The exit end face 50c emits the transmitted light. The reflective interface 50b tubularly extends in the optical axis direction from the entrance end face 50a to the exit end face 50c. The light homogenizer 50 propagates the incident light in the optical axis direction while the light is internally reflected in the total reflection by the reflective interface 50b defined between the light homogenizer 50 and the air.

In the light homogenizer 50, rays of the light incident upon the reflective interface 50b are reflected internally in the total reflection in the condition of a larger incident angle than a critical angle, because the rays travel from a medium (light homogenizer 50) with a high refractive index to a medium (air) with a low refractive index through the interface. The rays are reflected repeatedly within the light homogenizer 50 in the total reflection, for propagation in the optical axis direction.

A diameter of the entrance end face 50a of the light homogenizer 50 is approximately equal to that of the output fiber end 41e of the optical routing device 41. A diameter of the exit end face 50c is equal to that of an entrance end of the light guide device 43 of the endoscope 11. The entrance end face 50a of the light homogenizer 50 and the output fiber end 41e of the optical routing device 41 are thermally welded to one another in a unified form. The exit end face 50c is disposed in or near to the receptacle connector 42, and becomes opposed to the entrance end of the light guide device 43 when the second connection plug 28b of the endoscope 11 is coupled with the receptacle connector 42.

In FIG. 11, optical fibers positioned in respectively the areas a, b, c and d in the output fiber end 41e indicated by the phantom lines are assigned to the optical path of the input fiber ends 41a-41d. The optical fibers for the input fiber ends 41a-41d are distributed at the output fiber end 41e with local unevenness. Light incident through the input fiber ends 41a-41d is propagated in each of the optical fibers. There is no transmission of light between adjacent optical fibers. White light generated by the first light source units 31 exits from the output fiber end 41e through the areas a and b. Narrow band light N2 generated by the second light source unit 32 exits through the area c. Narrow band light N3 generated by the third light source unit 33 exits through the area d. In short, the light of the plural colors is distributed unevenly with the different areas. As a result, distribution of light amounts of light of the colors is uneven in a cross section of the light beam exited from the output fiber end 41e.

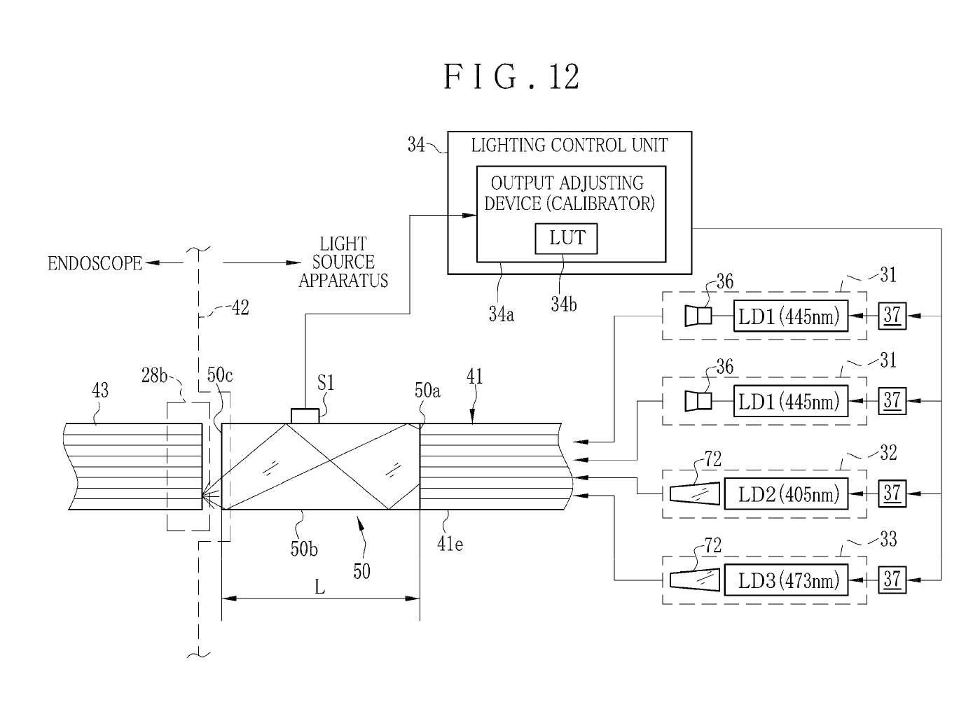

In FIG. 12, the light homogenizer 50 propagates incident light by the total reflection with the reflective interface 50b. An entrance position and exit position of the light are changed in a section perpendicular to the optical axis direction. This means that the position of the light varies in the radial direction during the propagation in the light homogenizer 50 in the total reflection. The variation of the position is effective in regularizing a light amount of the light exiting from the exit end face 50c of the light homogenizer 50 in the radial direction, because the unevenness of light of the plural colors at the output fiber end 41e is canceled. The number of the internal reflection increases according to greatness of a length L of the light homogenizer 50 in the optical axis direction, so that scattering of light owing to the variation of the position is remarkably effective.

The scattering of the light occurs for light of the plural colors from the light source units 31-33, so that the distribution of the light amounts of the colors is regularized. The light with the uniformity becomes incident upon the light guide device 43. The light is emitted by the lighting windows 22 of the endoscope 11 through the light guide device 43 toward an object of interest for illumination. No unevenness in the light amount or color occurs in an irradiated area of the object with the light.

The photo sensor S1 for light measurement is disposed on the reflective interface 50b of the light homogenizer 50. An example of the photo sensor S1 is a photo diode or other photoelectric conversion device for outputting an electric signal according to received light. The photo sensor S1 sends a light amount signal to the lighting control unit 34 according to an amount of the received light.

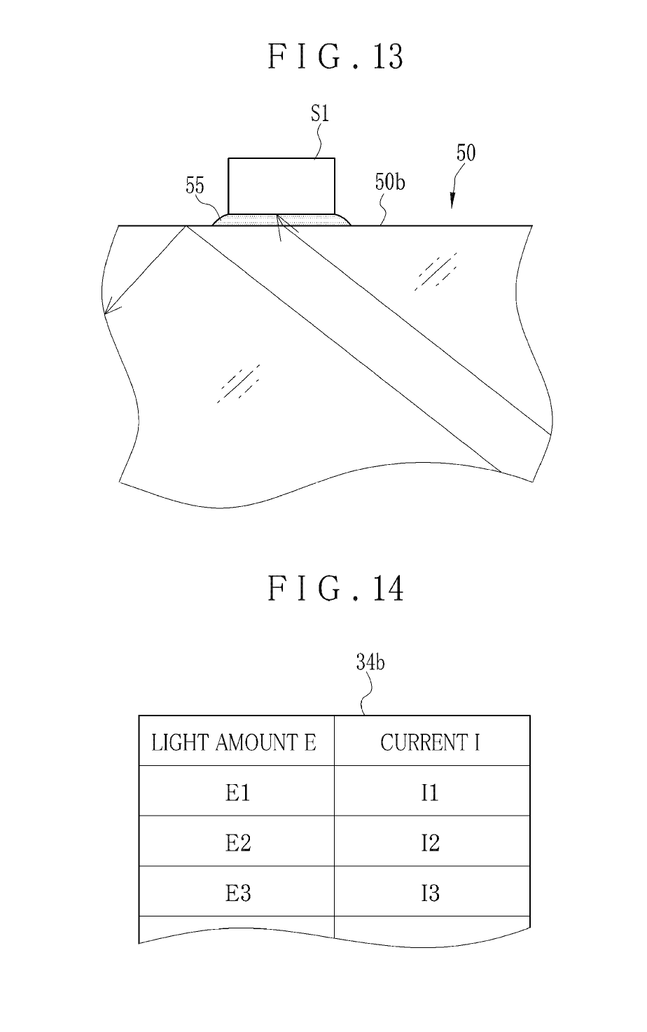

In FIG. 13, an adhesive agent 55 is used to attach the photo sensor S1 for light measurement to the reflective interface 50b or peripheral surface, and has a higher refractive index than a material for the light homogenizer 50. There are plural components of light incident upon the reflective interface 50b of the light homogenizer 50. A first one of the light components incident upon a point of the photo sensor S1 or at the adhesive agent 55 is not reflected by the reflective interface 50b in the total reflection, because the refractive index of the adhesive agent 55 is higher than that of the light homogenizer 50. The first component is passed through the reflective interface 50b and becomes incident upon the photo sensor S1. A second one of the light components incident upon a point offset from the photo sensor S1 (adhesive agent 55) is reflected by the reflective interface 50b in the total reflection on the condition of the incident angle higher than the critical angle. This is because the refractive index of the air is lower than that of the light homogenizer 50. The second component is propagated in the optical axis direction.

The photo sensor S1 for light measurement is sensitive to all wavelengths, namely sensitive to white light emitted by the first light source units 31 (mixture of the narrow band light N1 and fluorescence FL) and the narrow band light N2 and N3 from the second and third light source units 32 and 33. Light amounts of the light source units 31-33 can be measured only by use of the photo sensor S1.

In FIG. 12, an output adjusting device 34a (calibrator) is incorporated in the lighting control unit 34 for adjusting outputs of the light source units 31-33. The laser diodes LD1-LD3 in the light source units 31-33 change in a drive current for emitting light of a predetermined light amount according to an environmental condition and degradation with time. As the first light source units 31 have the phosphor 36, a light amount may be dropped by degradation of the phosphor 36 with time. The output adjusting device 34a adjusts the drive current of the laser diodes LD1-LD3 to regulate the light amount of the light source units 31-33 without changes even if there occurs a change in the environmental condition and degradation with time.

An LUT 34b (lookup table memory) is incorporated in the output adjusting device 34a, and stores information of a relationship between the light amounts E1, E2, E3 and so on of received light and drive currents I1, I2, I3 and so on for application to the laser diodes LD1-LD3. See FIG. 14. The light amount E is in a range of light receivable in the photo sensor S1 upon passage of light in the light homogenizer 50. The light amount of reception of the photo sensor S1 increases according to the light amount of light incident upon the light homogenizer 50 because of an increase in the light amount of light emitted by the light source units 31-33. The light amount of emission of the light source units 31-33 can be measured by measuring the light amount of the reception of the photo sensor S1.

The laser diodes LD1-LD3 are different from one another in the wavelength, type, and thus, a relationship between the light amount E and the drive current I. The first light source units 31 are different from the second and third light source units 32 and 33 in that the phosphor 36 is combined with the laser diode LD1. The LUT 34b is provided for each of the light source units 31-33. The first light source units 31 emit the narrow band light N1 generated by the laser diode LD1 and the fluorescence FL mixed therewith. The LUT 34b for the first light source units 31 stores a relationship between the drive current I and the light amount E of the white light obtained by mixture of the narrow band light N1 and the fluorescence FL. The LUT 34b for the second and third light source units 32 and 33 stores a relationship between the drive current I of the laser diode LD2 and the light amount E of the narrow band light N2, and a relationship between the drive current I of the laser diode LD3 and the light amount E of the narrow band light N3.

The output adjusting device 34a adjusts the drive currents of the laser diodes LD1-LD3 by output adjustment (calibration), and updates the drive current I stored in the LUT 34b for a value after the adjustment.

The output adjusting device 34a operates for output adjustment upon starting up the light source apparatus 13, for example, when a power source for the light source apparatus 13 is turned on. It is possible to operate the output adjusting device 34a periodically, for example, once per a day, once per a week, and once per a month. Also, the output adjusting device 34a can be caused to adjust outputs manually by use of the console unit 15.

Also, it is possible to operate the output adjusting device 34a at a small regular period while the light source apparatus 13 is not used, for example, once per a minute.

For the output adjustment, the laser diodes LD1-LD3 are driven with the drive current I of the LUT 34b before the output adjustment, to turn on the light source units 31-33 one after another. The output adjusting device 34a checks whether a reception light amount E according to the drive current I is obtained according to the light amount signal from the photo sensor S1 for light measurement in relation to a specific one of the light source units 31-33. If there is a difference between the measured light amount E and the reference light amount E in the LUT 34b, the drive current I is adjusted to set the measured light amount E equal to the reference light amount E. The adjusted drive current I is written to the LUT 34b for updating the data.

A shutter (not shown) is disposed in the receptacle connector 42 for preventing leakage of light from the exit end face 50c of the light homogenizer 50 in the course of output adjustment without connecting the endoscope 11 to the light source apparatus 13. The shutter is biased toward a closed position from an open position by a spring or the like when in an initial position. When the second connection plug 28b of the endoscope 11 is connected with the receptacle connector 42, the shutter becomes open by the pressure of the second connection plug 28b. The receptacle connector 42 can be light-tight even when the output adjustment is carried out only with the light source apparatus 13.

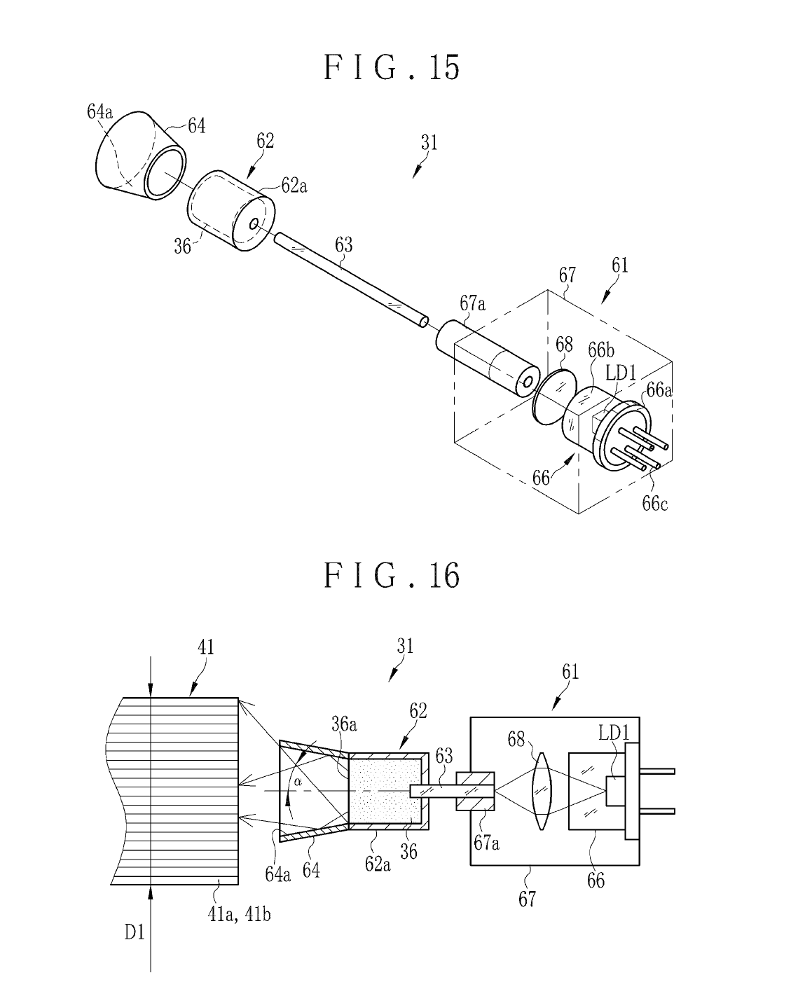

In FIGS. 15 and 16, each of the first light source units 31 includes a laser source 61, a wavelength converter 62 of phosphor conversion, fiber optics 63 of a single fiber, and a divergence angle corrector 64. The fiber optics 63 guide the light from the laser source 61 to the wavelength converter 62. The divergence angle corrector 64 is mounted on an end of the wavelength converter 62. The laser source 61 is in a receptacle form, and includes a light emitting device 66 or laser diode LD1, and a source housing 67 for containing the light emitting device 66. A fiber coupler 67a is provided in the source housing 67 for connection of one end of the fiber optics 63. A condenser lens 68 is incorporated in the source housing 67.

The light emitting device 66 includes a support disk 66a as a stem, the laser diode LD1, a transparent cap 66b, and leads 66c or lines. The laser diode LD1 is a semiconductor chip, and attached to a surface of the support disk 66a. The transparent cap 66b is a cylindrical part of resin, and covers the laser diode LD1. The leads 66c extend from a second surface of the support disk 66a.

The laser diode LD1 includes a P layer of a P type semiconductor and an N layer of an N type semiconductor mounted on the P layer with an active layer, which emits laser light according to laser oscillation. The laser light generally travels straight, but is diverging light of which a diameter of a beam shape increases conically from the emission point. The laser light is condensed by the condenser lens 68 at the entrance end of the fiber optics 63.

An exit end of the fiber optics 63 is coupled with the wavelength converter 62. A container 62a for protection is a cylindrical part of a light-tight property, and filled with the phosphor 36 to constitute the wavelength converter 62. A fiber hole is formed at the center of the phosphor 36 and receives entry of the fiber optics 63. A ferrule (not shown) for connection is mounted on an end of the fiber optics 63, which are entered in the phosphor 36 together.

The phosphor 36 includes phosphor material of a powder form, and binder of resin in which the phosphor material is dispersed and hardened. Emission points of the fluorescence FL upon excitation are disposed on the entirety of the exit end face of the phosphor 36 because of the dispersion. Laser light transmitted through the phosphor 36 is diffused owing to the effect of light scattering of the binder, so that the emission points of the fluorescence FL are disposed on the entirety of the exit end face.

Light emitted by the phosphor 36 is diverging light traveling from the emission points conically in a manner similar to the laser diode LD1. An area of the emission point and a divergence angle of the phosphor 36 are larger than those of the laser diode LD1.

The phosphor 36 has an exit end face 36a. The divergence angle corrector 64 is disposed downstream of the wavelength converter 62 for correcting a divergence angle of the light emitted by the exit end face 36a. The divergence angle corrector 64 is formed cylindrically from opaque material, and reduces the divergence angle by limiting passage of the diverging light from the phosphor 36. An inner surface 64a of the divergence angle corrector 64 is coated with reflective material, and is a mirror surface for the divergence angle corrector 64 to operate as a reflector. The light is reflected by the inner surface 64a and is propagated in the optical axis direction. A loss in the light transmission is low because the absorption of light is low with the inner surface 64a.

Inclination angles of the divergence angle corrector 64 with respect to the transverse direction and optical axis direction are predetermined in consideration of the diameter D1 of the input fiber ends 41a and 41b. The diameter and the inclination angles are so determined that a spot diameter of a beam from the first light source units 31 to the input fiber ends 41a and 41b is substantially equal to the diameter D1 of the input fiber ends 41a and 41b.

A divergence angle α in FIG. 16 is determined according to a numerical aperture (NA) of optical fibers as elements of a fiber bundle such as the optical routing device 41, the light guide device 43 of the endoscope 11, and the like. As is well-known in the art, an optical fiber includes a core with a high refractive index, and a cladding with a low refractive index. The incident light upon entry in the optical fiber travels in the optical axis direction. It is necessary to make light incident upon an entrance end face to satisfy the condition of total reflection for the purpose of the propagation.

NA is a value of ability of an optical fiber for condensing light, and is defined as NA=sin θmax wherein θmax is a maximum reception angle. NA increases according to an increase in the maximum reception angle ° max. If an incident angle of light incident in the optical fibers is equal to or smaller than the maximum reception angle θmax, total reflection occurs on an interface between the core and cladding in the optical fiber. The incident light travels in the optical axis direction. If the incident angle becomes larger than the maximum reception angle θmax, the incident light cannot be propagated, because of passage without total reflection. There occurs a loss in the transmission of light. To reduce the loss in the light, the divergence angle corrector 64 regulates the divergence angle α of the light flux from the first light source units 31 equal to or lower than the maximum reception angle θmax.

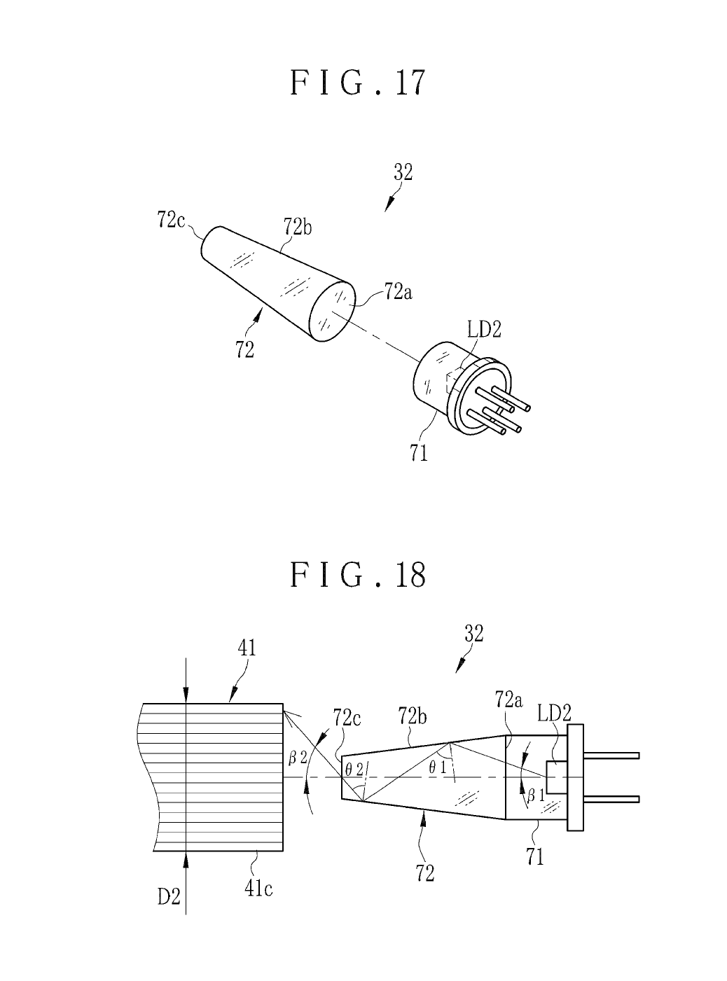

In FIGS. 17 and 18, the second light source unit 32 includes a light emitting device 71 or laser diode, and a divergence angle corrector 72. The light emitting device 71 has the laser diode LD2, and is structurally the same as the light emitting device 66 in the first light source units 31. The divergence angle corrector 72 is a light guide device, formed from transparent material, and shaped conically near to a rod form. The divergence angle corrector 72 can be called a light pipe or light tunnel. The divergence angle corrector 72 of a total reflection type has an entrance end face 72a, a reflective interface 72b or peripheral surface, and an exit end face 72c. In a manner similar to the light homogenizer 50, the entrance end face 72a receives entry of light. The reflective interface 72b directs the light in the optical axis direction by total reflection. The exit end face 72c emits the light. The entrance end face 72a of the divergence angle corrector 72 is thermally welded with a tip of the light emitting device 71 for a unified form.

The divergence angle corrector 72 is so shaped that the reflective interface 72b is tapered from the entrance end face 72a toward the exit end face 72c with a decreasing diameter, or in a shape of a frustum of a cone. In FIG. 17, a second reflection angle θ2 of the incident light is smaller than a first reflection angle θ1. A reflection angle θ decreases gradually by repeated reflection on the reflective interface 72b. The decrease in the reflection angle θ means an increase in the divergence angle. The divergence angle β2 of the light emitted by the laser diode LD2 is increased to a divergence angle β2 by operation of the divergence angle corrector 72.

Effect of enlarging the divergence angle increases according to an increase in the length of the divergence angle corrector 72 in the optical axis direction, because the number of times of reflection on the reflective interface 72b increases. Also, effect of enlarging the divergence angle per one time of reflection increases according to an increase in an inclination angle of the reflective interface 72b.

The length of the divergence angle corrector 72 in the optical axis direction, its inclination angle of a reflection surface with respect to the optical axis direction, and an interval between a distal end of the divergence angle corrector 64 and the input fiber end 41c are determined in consideration of the numerical aperture (NA) of optical fibers in the light guide device 43 of the endoscope 11 and the diameter D2 of the input fiber end 41c. This is similar to the divergence angle corrector 64 of the first light source units 31. Specifically, the length, inclination angle and interval of the divergence angle corrector 72 are determined so that the divergence angle 132 (indicated as a half width at half maximum) becomes equal to the maximum reception angle θmax (see FIG. 16) corresponding to the numerical aperture (NA) of the optical fibers, and that a spot diameter of a light flux incident upon the input fiber end 41c is equal to the diameter D2 of the input fiber end 41c.

If the divergence angle β2 is equal to or less than a maximum reception angle θmax (of FIG. 16), a loss in the optical transmission in the optical fiber is small because the light incident upon the optical fiber satisfies the total reflection condition, as has been described with the divergence angle corrector 64 of the first light source units 31. When the divergence angle β2 is maximized, a distribution angle of light from the lighting windows 22 of the endoscope 11 is increased to enlarge a lighting area in a region of interest in the body. A spot diameter of the light is set equal to a diameter of the input fiber end 41c. Efficiency in the light transmission can be higher because a higher number of optical fibers included in the input fiber end 41c can receive light.

For the third light source unit 33, the second light source unit 32 is repeated but with a difference in that a light emitting device 76 or laser diode LD3 of FIG. 10 is provided in place of the light emitting device 71. A divergence angle of the light emitting device 76 of the third light source unit 33 is enlarged by the divergence angle corrector 72 to be equal to the maximum reception angle θmax of optical fibers in the input fiber end 41d.

Unlike the first light source units 31, the second and third light source units 32 and 33 are devices without the phosphor 36. There is no large difference in the divergence angle between the second and third light source units 32 and 33. If there is a difference in the divergence angle between the light emitting devices 66 and 71, their correction amounts are determined so as to compensate for the difference, for example, by changing inclination angles of the reflective interface 72b of the divergence angle corrector 72.

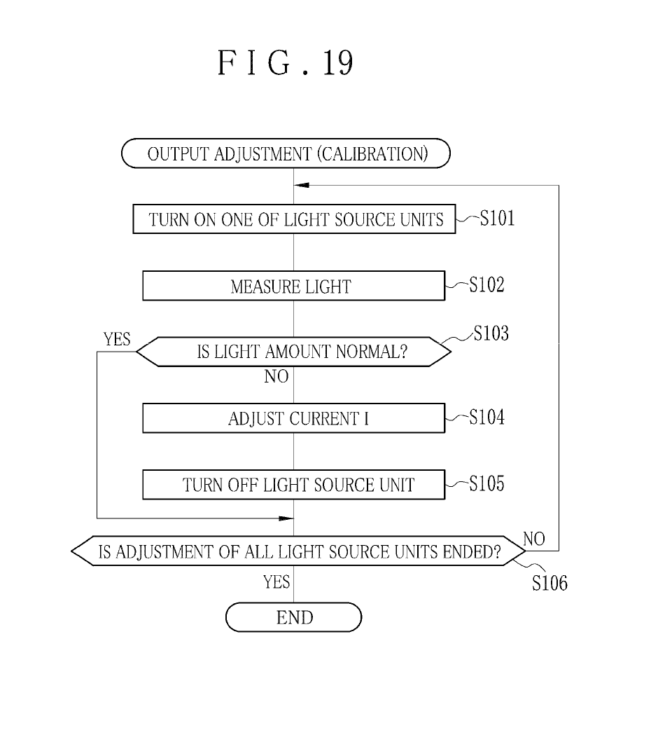

The operation of the embodiment is described by referring to a flow of FIG. 19. When a power source of the light source apparatus 13 is turned on, the light source apparatus 13 is started up. In response, the output adjustment is carried out in the light source apparatus 13.

The output adjusting device 34a turns on the light source units 31-33 one after another to adjust those serially. The two of the first light source units 31 are adjusted in output adjustment discretely from one another. The output adjusting device 34a reads the drive current I predetermined in the LUT 34b, and turns on one of the first light source units 31 to be adjusted. See the step S101.

Light from the first light source unit 31 travels through the optical routing device 41 and enters the light homogenizer 50. The photo sensor S1 receives part of light incident upon the light homogenizer 50, and outputs a light amount signal to the output adjusting device 34a. The output adjusting device 34a measures the light amount E of the first light source unit 31 according to the light amount signal from the photo sensor 51 in the step S102. The output adjusting device 34a refers to the LUT 34b and checks whether a measured light amount is equal to the reference light amount E corresponding to the drive current I in the step S103.

If the measured light amount is not equal to the predetermined light amount E (no in the step S103), then the output adjusting device 34a adjusts the drive current to set the measured light amount equal to the predetermined light amount E (S104). For example, if the measured light amount is lower than the predetermined light amount E, then the drive current is increased. If the measured light amount is higher than the predetermined light amount E, then the drive current is decreased. The output adjusting device 34a updates the LUT 34b by rewriting the drive current I by use of an adjusted drive current. After the adjustment, the first light source unit 31 is turned off (S105). If the measured light amount is equal to the predetermined light amount E (yes in the step S103), then the first light source unit 31 is turned off without updating the LUT 34b.

Similarly, the output adjusting device 34a operates for output adjustment of the remainder of the first light source units. 31 and the second and third light source units 32 and 33. When the adjustment of the light source units 31-33 is completed (yes in the step S106), the output adjustment is ended.

Also, it is possible in the step S103 to detect coincidence between the measured light amount and the predetermined light amount E not only if the measured light amount is exactly equal to the predetermined light amount E, but also if the measured light amount is in a predetermined range including the predetermined light amount E.

The light homogenizer 50 is disposed downstream of the optical routing device 41 for aligning optical paths from the light source units 31-33. Light emitted by the light source units 31-33 enters the light homogenizer 50. As the photo sensor S1 is disposed on the light homogenizer 50, the output adjustment can be carried out for all the light source units 31-33 only with the single photo sensor S1.

For diagnosis, the endoscope 11 is connected to the processing apparatus 12 and the light source apparatus 13. A power source for the processing apparatus 12 and the light source apparatus 13 is turned on to start up the endoscope system 10.

The elongated tube 16 of the endoscope 11 is entered in a gastrointestinal tract of a body, to start imaging. In FIG. 8A for the normal imaging mode (color imaging mode), the first light source units 31 are turned on to apply white light to an object for imaging, as a mixture of the narrow band light N1 emitted by the laser diode LD1 and the fluorescence FL emitted by the phosphor 36. In FIG. 8B for the vessel enhancement mode, the first and second light source units 31 and 32 are turned on to apply white light and the narrow band light N1 to the object for imaging. In FIG. 8C for the oxygen saturation monitoring mode, the first and third light source units 31 and 33 are turned on to apply white light and the narrow band light N3 to the object for imaging.

The lighting control unit 34 accesses the LUT 34b updated by the output adjustment, determines conditions for driving the light source units 31-33, and turns on those. According to a control signal from the processing apparatus 12, the lighting control unit 34 sets light amounts of the light source units 31-33 for exposure control. As the light source units 31-33 are adjusted in the output adjustment, light of a normal light amount can be stably obtained irrespective of changes in the conditions or degradation with time.

In the present invention, the light source apparatus 13 has the photo sensor S1 for light measurement. Output adjustment of the light source units 31-33 can be carried out inside the light source apparatus 13. As the photo sensor S1 is disposed on the reflective interface 50b of the light homogenizer 50, it is possible to prevent vignetting which would occur in disposing the photo sensor in an optical path.

The light homogenizer 50 is an optical element for regularizing distribution of the light amount of light emitted by the light source units 31-33, and is not specialized for measuring the light amount. Unlike the beam splitter of the patent document JP-A 2011-183099, it is unnecessary to use a specialized component for measuring the light amount in addition to the photo sensor S1. A manufacturing cost can be set low because there is no increase in the number of the parts or no complication of the structure. There is no increase in an inner space due to addition of a specialized component. It is easy to mount the photo sensor S1, which is only attached to the light homogenizer 50 by the adhesive agent 55.

The beam splitter according to the patent document has a drawback in that a loss of light may occur considerably in disposing a specialized component for light measurement in an optical path. However, the construction of the invention is free from such a problem.

The output adjustment of the light source units 31-33 is remarkably important in the additional use of the second and third light source units 32 and 33 for the special imaging with the first light source units 31. In the vessel enhancement mode, the first light source units 31 for the white light and the second light source unit 32 for the narrow band light N2 are used. Contrast of the blood vessels in the image is determined according to a light amount ratio between the white light and the narrow band light N2. It is necessary to maintain the light amount ratio at a normal level between the white light and the narrow band light N2 to optimize the contrast of the blood vessels. The exposure is controlled in maintaining the light amount ratio suitably.

In the oxygen saturation monitoring mode, the first light source units 31 for white light and the third light source unit 33 for narrow band light N3 are used. Images are obtained by use of the white light and the narrow band light N3, so that arithmetic operation is carried out between the images. If a light amount ratio between the first and third light source units 31 and 33 is not normal in the arithmetic operation, reliability of the determined oxygen saturation level is low. The light amount ratio should be maintained normally. Accordingly, output adjustment is essentially important for the purpose of maintaining the light amount ratio.

In use of the light source unit for the special light imaging, the purpose of the output adjustment is not only for regularizing the brightness of the light but for optimizing the condition of the special light imaging. The output adjustment of the light source unit is more useful in the light source apparatus 13 with a function for the special light imaging than in that without a function for the special light imaging, and thus is used more frequently in the former than in the latter. Structural simplification of a light source unit used frequently is effective in lowering a factor of occurrence of failure. Thus, stability of the light source unit can be higher. In conclusion, simplification of the structure for the output adjustment in the light source unit according to the present invention is particularly effective in the light source apparatus 13 with the function for the special light imaging.

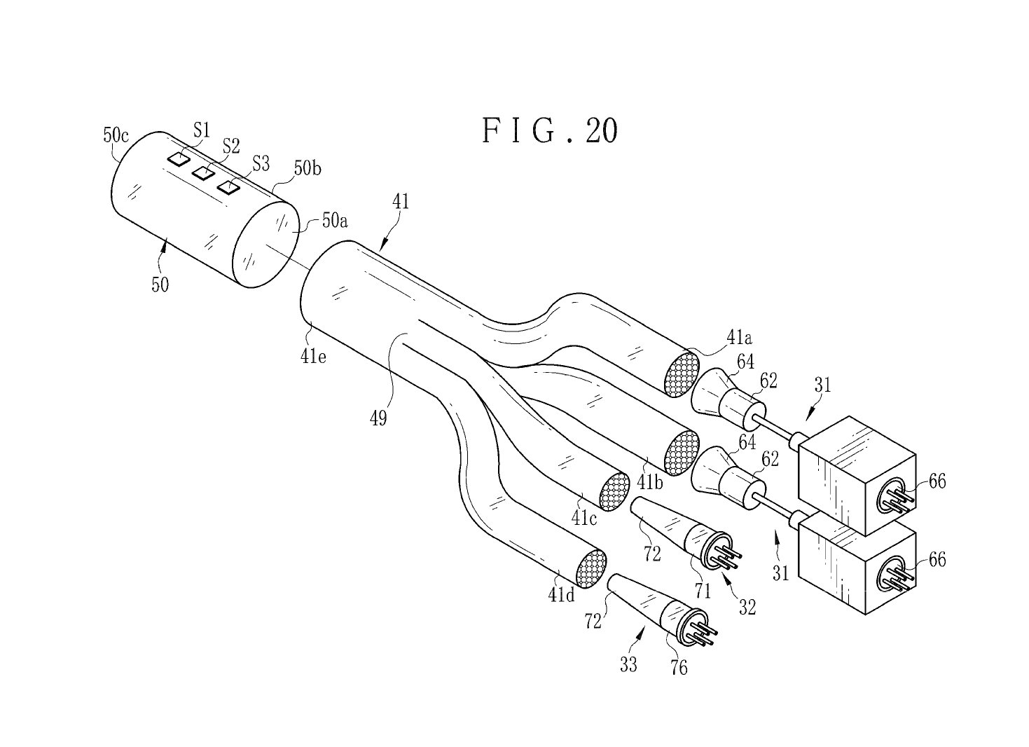

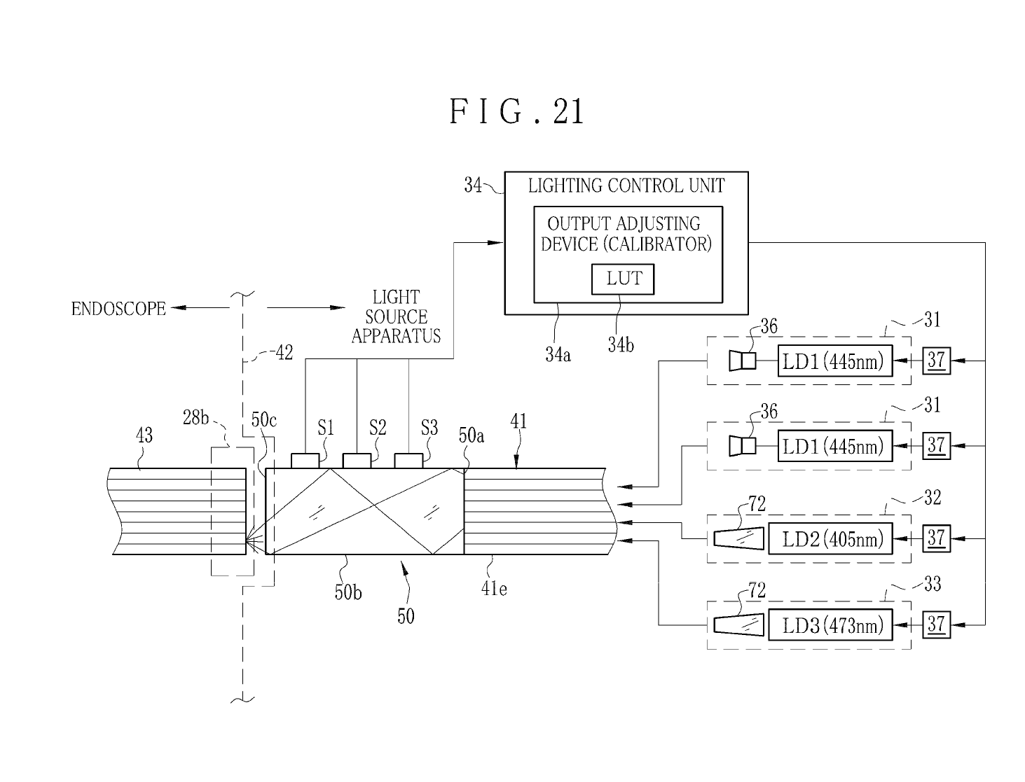

In FIGS. 20 and 21, another preferred embodiment is illustrated, in which a plurality of photo sensors for light measurement are incorporated. Elements similar to those of the above embodiment are designated with identical reference numerals.

Photo sensors S2 and S3 are added to the photo sensor S1 in the light homogenizer 50, which operates in the three-sensor structure. The photo sensors S1-S3 are attached by the adhesive agent 55 of the high refractive index similar to the first embodiment.

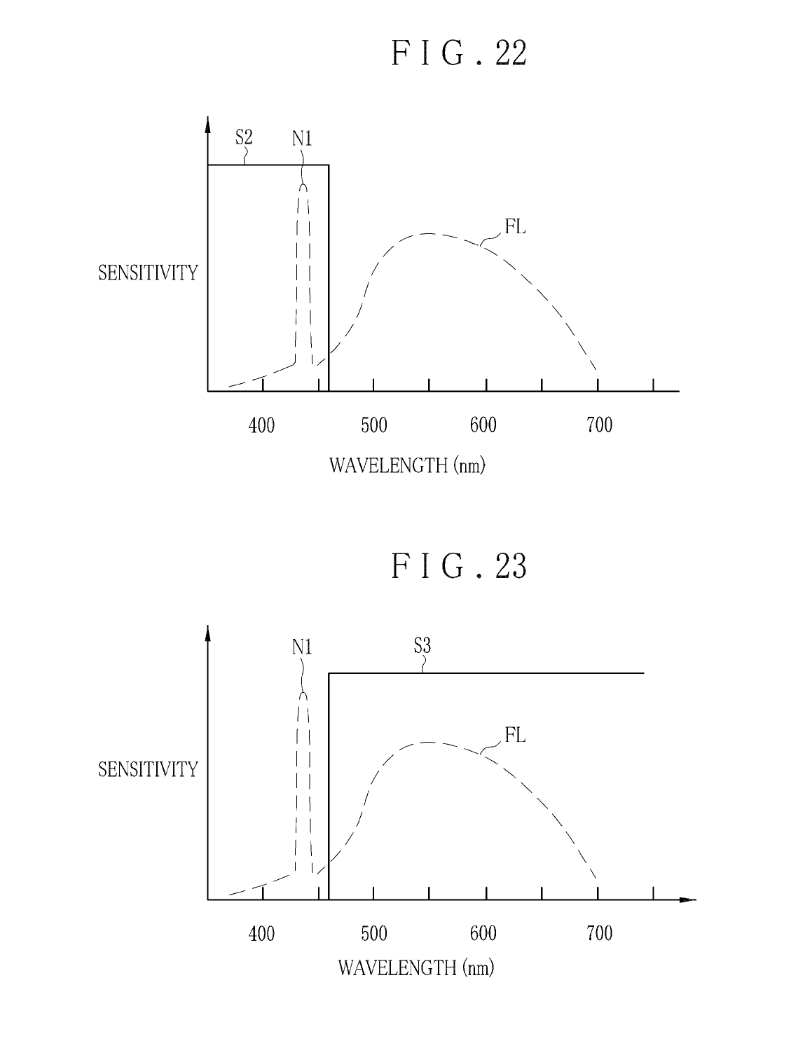

The photo sensors S2 and S3 measure white light from the first light source units 31 by separation into the narrow band light N1 and fluorescence FL. The photo sensor S2 is sensitive only to light of a wavelength equal to or smaller than approximately 460 nm and not sensitive to light of larger wavelengths. See FIG. 22. Thus, the photo sensor S2 is sensitive to the narrow band light N1 of 440 plus or minus 10 nm but not to the fluorescence FL. The photo sensor S2 is constituted by a cut filter for cutting light of a wavelength of approximately 460 nm or larger on an imaging surface of a sensor similar to the photo sensor S1.

In contrast, the photo sensor S3 is sensitive only to light of a wavelength equal to or larger than approximately 460 nm and not sensitive to light of smaller wavelengths. See FIG. 23. Thus, the photo sensor S3 is sensitive to the fluorescence FL but not to the narrow band light N1. In a manner similar to the photo sensor S2, the photo sensor S3 is constituted by a sensor the same as the photo sensor S1, and a cut filter disposed on an imaging surface of the sensor for absorbing light of a wavelength equal to or less than approximately 460 nm.

It follows that the use of the photo sensors S2 and S3 is effective in selectively recognizing the reasons of a drop of the light amount of the first light source units 31 between degradation of the phosphor 36 and degradation of the laser diode LD1.

The LUT 34b of the second embodiment stores the information according to the first embodiment and also data of first and second relationships. The first relationship is between the drive current I of the laser diode LD1 and a light amount Ea of the narrow band light N1 included in the white light from the first light source units 31. The second relationship is between the drive current I of the laser diode LD1 and a light amount Eb of the fluorescence included in the white light. Also, the LUT 34b stores the information of a normal range for each light amount ratio of the narrow band light N1 and the fluorescence FL to the white light.

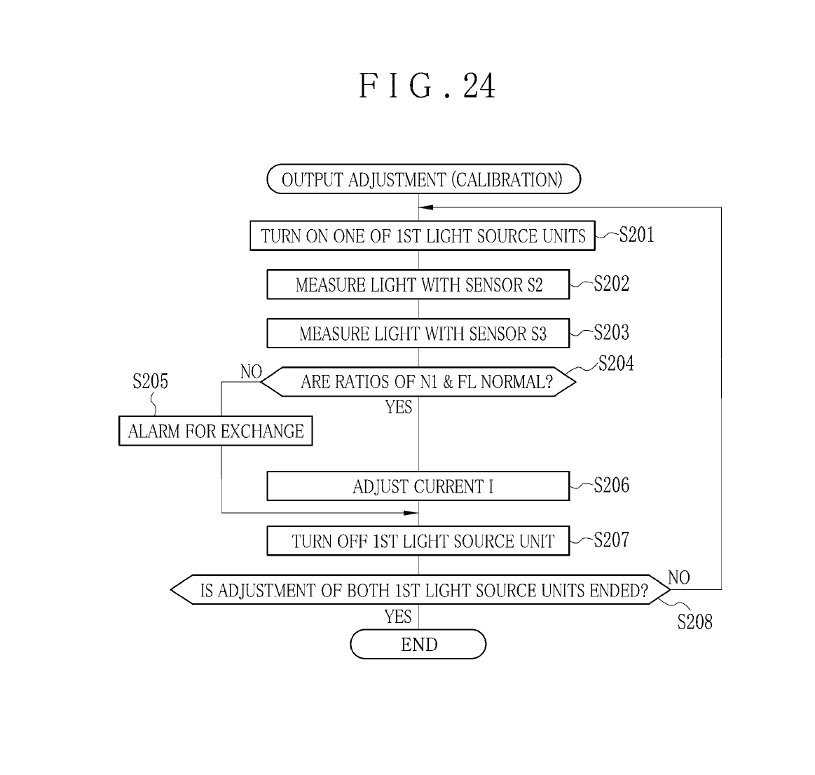

The output adjustment by use of the photo sensors S2 and S3 for light measurement is carried out according to a flow of FIG. 24. At first, the output adjusting device 34a turns on one of the first light source units 31 to be adjusted (S201). White light emitted by the first light source unit 31 passes through the optical routing device 41 and enters the light homogenizer 50. The photo sensor S2 sends a light amount signal to the output adjusting device 34a according to a light amount of received narrow band light N1 in the white light. The output adjusting device 34a measures the light amounts of the narrow band light N1 and the fluorescence FL according to the light amount signals from the photo sensors S2 and S3 in the steps S202 and S203.

The output adjusting device 34a refers to the LUT 34b and checks whether each of the light amount ratios of the narrow band light N1 and the fluorescence FL is in a normal range in the step S204. Even if measured light amounts of the narrow band light N1 and the fluorescence FL are lower than the predetermined light amounts Ea and Eb, the light amount ratios are likely to be in the normal range. Then it is supposed that degradation of the laser diode LD1 and the fluorescence FL becomes more serious in a correlated manner. For this situation, the drive current I of the laser diode LD1 may be increased to adjust the light amount of the first light source unit 31 properly. If the output adjusting device 34a judges that the light amount ratios of the narrow band light N1 and the fluorescence FL are in the normal range (yes in the step S204), then the output adjusting device 34a adjusts the drive current I to update the LUT 34b in the step S206.

If it is judged that the light amount ratios of the narrow band light N1 and the fluorescence FL are not in the normal range (no in the step S204), the output adjusting device 34a generates an alarm signal for encouraging exchange of the first light source unit 31 (S205). There has occurred a difference in the degradation between the laser diode LD1 and the phosphor 36 if the light amount ratios of the narrow band light N1 and the fluorescence FL are not in the normal range. It is supposed that good white color with appropriate color balance cannot be obtained only by adjusting the drive current I for the laser diode LD1.

In the alarm processing, the output adjusting device 34a sends an alarm signal to the processing apparatus 12 for displaying an alarm message on the display panel 14 in connection with the processing apparatus 12. Also, an indicator lamp (not shown) of the light source apparatus 13 can be used and turned on continuously or in a winking manner.

An example of alarm message for the exchange is an exchange of the entirety of the first light source unit 31. Also, an alarm message may be an exchange of only the phosphor 36 typically when the output adjusting device 34a evaluates the light amount ratios of the narrow band light N1 and the fluorescence FL and judges that only the phosphor 36 should be exchanged. An example of criteria of judging that only the phosphor 36 should be exchanged is in that the narrow band light N1 of the predetermined light amount Ea is obtained but that the fluorescence FL of the predetermined light amount Eb is not obtained. The output adjusting device 34a refers to the LUT 34b and operates for this judgement.

The first one of the first light source units 31 after the adjustment is turned off in the step S207. A second one of the first light source units 31 is adjusted in the similar manner. The output adjustment is terminated when both of the first light source units 31 are adjusted finally. See the step S208.

In the present embodiment, the narrow band light N1 and the fluorescence FL in the white light are separated for the first light source units 31 with the phosphor by use of the photo sensors S2 and S3, to measure the light amounts. The feature of the invention is very suitable for this measurement of the light amounts in comparison with the construction of the patent document JP-A 2011-183099.

The method of the patent document with the beam splitter has a shortcoming in a very complicated structure, because part of white light from the beam splitter in the optical path is directed to a photo sensor for light measurement, and a structure for color separation of the directed white light must be disposed upstream of the photo sensor, such as a turret filter. Also, vignetting remarkably occurs because of a plurality of photo sensors disposed in the optical path with differences in spectral sensitivity. In the present invention, however, the number of the photo sensors in combination with the light homogenizer 50 can be increased, so that no complication of the structure will occur. The reflective interface 50b of the light homogenizer 50 has a sufficiently large area for disposing the plural photo sensors, so that it is easy to keep a sufficient space for the disposition.

There are other methods of utilizing the plural photo sensors for light measurement in a manner different from the above embodiment. Specifically, the light source units 31-33 are different in the wavelength of the emission. It is possible to dispose photo sensors for respectively the light source units with spectral sensitivity suitable for respectively the wavelengths of the light source units 31-33. Thus, measurement of light amounts at high precision is possible. The output adjustment can be carried out appropriately.

Also, it is possible to provide a plurality of photo sensors of the same type for light measurement, and to determine an average of light amount signals from the photo sensors for measuring the light amount. Such photo sensors can be the same as the photo sensor S1 of the first embodiment. It is possible to increase precision in the measurement in comparison with the use of the single photo sensor of the same type.

Also, the photo sensor for light measurement can be used for the purpose other than the output adjustment (calibration) of the light source units. For example, light amounts of the B, G and R components in the white light are measured as disclosed in the patent document, to adjust the signal gains of the colors of the image signal according to the measured light amounts, for the purpose of adjusting the color balance of the image. To this end, B, G and R photo sensors sensitive to respectively B, G and R colors can be provided to input detection signals from those to the processing apparatus 12. Also, the use of the photo sensor can be for a purpose other than the gain adjustment.

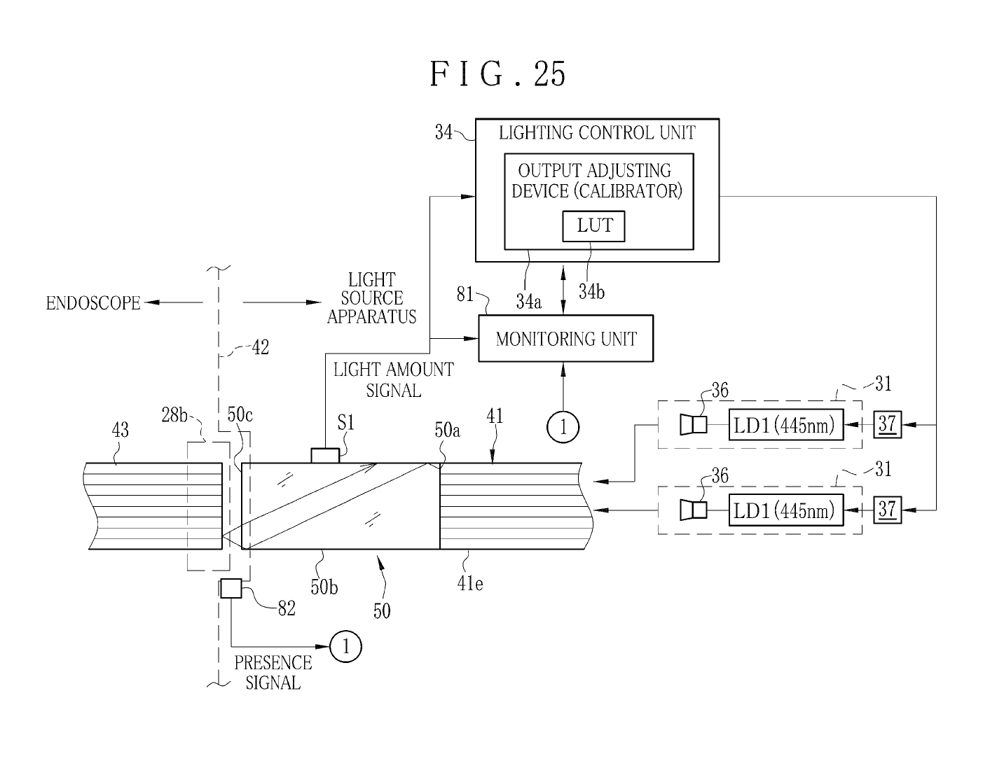

In FIGS. 25, 26, 27 and 28, a third preferred embodiment is illustrated, in which the photo sensor S1 for light measurement is used for testing a mounted condition of the proximal connector 28 of the endoscope 11. Elements similar to those of the above embodiment are designated with identical numerals.

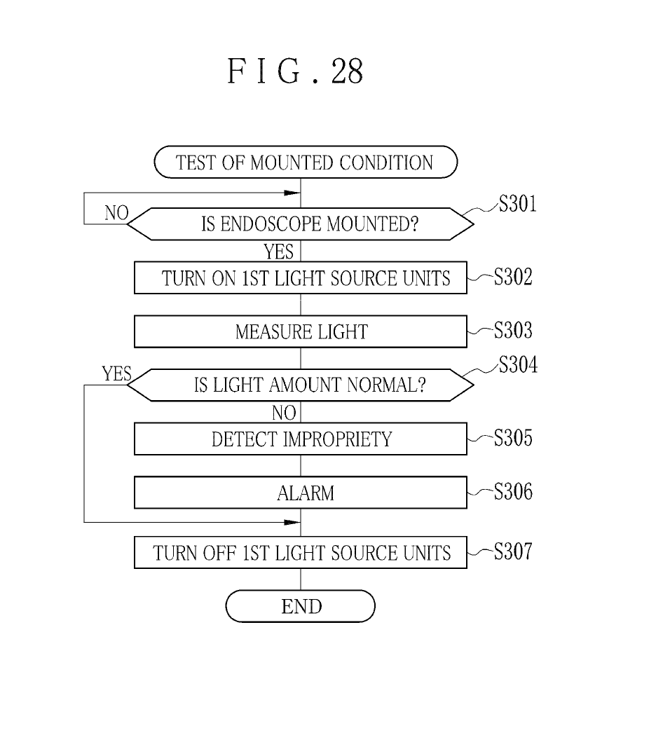

In FIG. 25, the first embodiment is repeated in the third embodiment but with a difference in that a monitoring unit 81 and a presence sensor 82 are added. The presence sensor 82 checks whether the endoscope 11 is present on the light source apparatus 13 in the step S301 in FIG. 28. While the light source apparatus 13 is driven, the presence sensor 82 monitors a mounted condition of the light source apparatus 13 to the endoscope 11.

The presence sensor 82 is a photo sensor, such as a photo diode, and disposed in the receptacle connector 42. The presence sensor 82 detects entry of the second connection plug 28b of the endoscope 11 in the receptacle connector 42, and sends a presence signal to the monitoring unit 81.

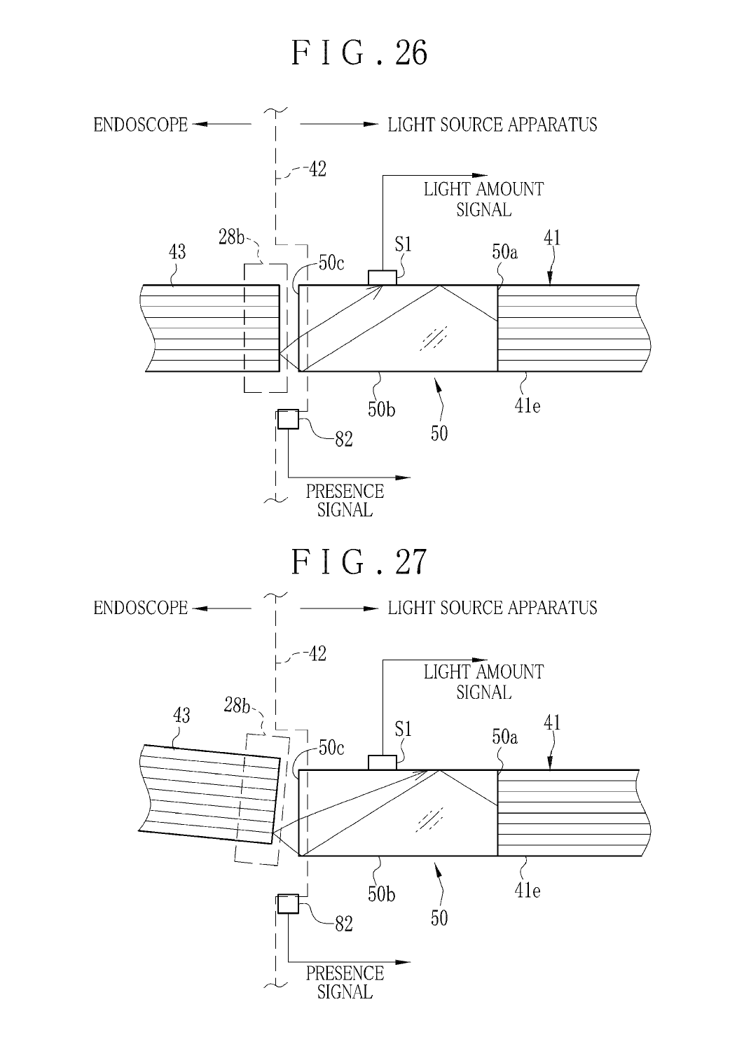

The monitoring unit 81, upon receiving the presence signal, causes the lighting control unit 34 to turn on the first light source units 31 in the step S302. As illustrated in FIGS. 26 and 27, white light from the first light source units 31 becomes incident upon the light homogenizer 50 to enter the entrance end face of the light guide device 43. The white light is transmitted from the light homogenizer 50 to the light guide device 43. However, a small part of the white light is reflected by the entrance end face of the light guide device 43, and enters the exit end face 50c of the light homogenizer 50 again as reflected light.

In FIG. 26, the second connection plug 28b is directed properly. In FIG. 27, the second connection plug 28b is improperly mounted with an inclination. An entrance end face of the light guide device 43 is differently oriented between the states of FIGS. 26 and 27. An angle of entrance of the reflected light to the light homogenizer 50 is changed. If the second connection plug 28b is improperly mounted, the amount of leaked light outside the receptacle connector 42 is likely to increase without entry in the light guide device 43. An amount of the reflected light for reentry to the light homogenizer 50 may decrease. Thus, a difference occurs in the amount of light received by the photo sensor S1 between the proper and improper mounted conditions.

An internal memory (not shown) in the monitoring unit 81 stores a normal range of a light amount of received light of the photo sensor S1 in the properly mounted condition. The monitoring unit 81 measures a light amount of the photo sensor S1 according to the signal output by the photo sensor S1 in the step S303. The monitoring unit 81 checks whether the light amount is within the normal range in the step S304, and if the light amount is within the normal range (yes in the step S304), judges that the second connection plug 28b is mounted properly. The first light source units 31 are turned off in the step S307 to terminate the function of testing the mounted condition.

If the light amount is not in the normal range (no in the step S304), then the monitoring unit 81 judges that the mounted condition is improper in the step S305. Then an alarm signal is generated to notify the impropriety of the mounted condition in the step S306. In a manner similar to the alarming in the second embodiment (FIG. 24), the alarm signal is input to the processing apparatus 12 for driving the display panel 14 to display an alarm message. Also, an indicator of the light source apparatus 13 may be turned on continuously or in a winking manner. A user or operator of the endoscope 11 can be informed of the impropriety of the mounted condition. After the alarming, the first light source units 31 are turned off in the step S307.

The function of testing the mounted condition is according to the reflected light from the light guide device 43 of the endoscope 11. The photo sensor S1 for the purpose is disposed on the light homogenizer 50 upstream of the light guide device 43 and can detect a change in the reflected light due to a difference in the mounted condition. In conclusion, the photo sensor S1 disposed in this manner can be utilized for testing the mounted condition of the endoscope 11 in addition to output adjustment of the light source units 31-33. The importance of disposing the photo sensor on the light homogenizer 50 upstream of the light guide device 43 is very high in the light source according to the invention.

If the method with the beam splitter in the patent document JP-A 2011-183099 is used, it is impossible to test the mounted condition. This is because the beam splitter has a reflection film which reflects part of incident light and guides this to a photo sensor for light measurement. The reflected light from the exit side toward the reflection film cannot be guided to the photo sensor. Also, the known method is not preferable because of creation of vignetting, as the photo sensor is disposed in the optical path.

In the present embodiment, the first light source units 31 are turned on together for testing the mounted condition. However, only one of the first light source units 31 may be turned on for testing the mounted condition.

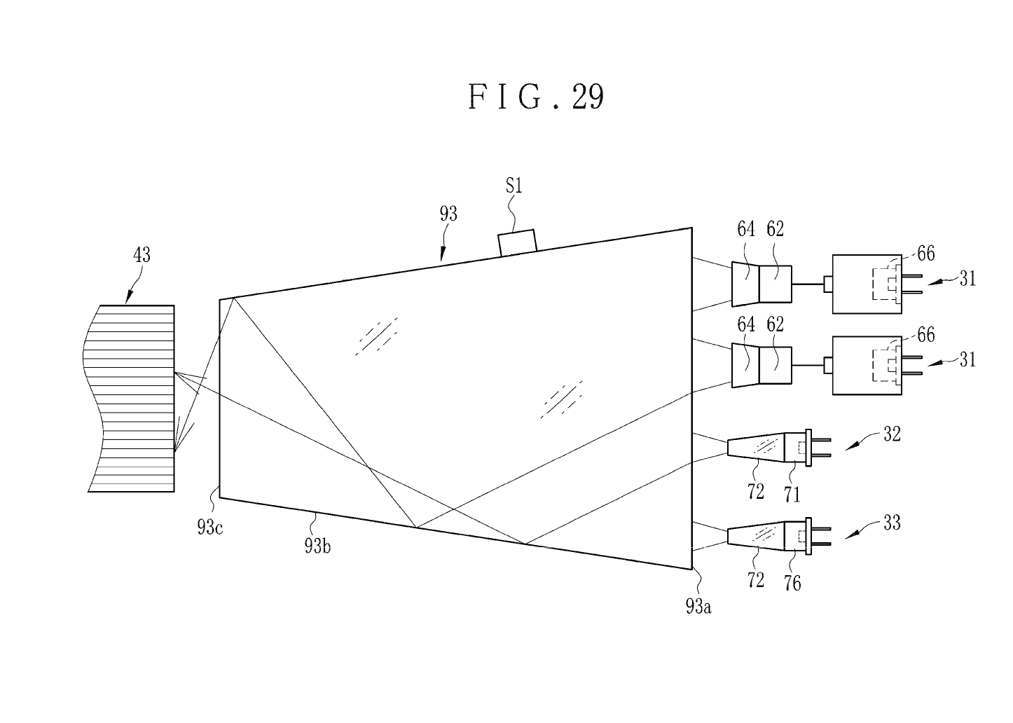

In the above embodiment, the light homogenizer 50 is a separate device from the optical routing device 41. In FIG. 29, another preferred structure is illustrated, in which a light homogenizer 93 (integrating device) or optical coupler is a single device including functions of the light homogenizer 50 and the optical routing device 41.

In FIG. 29, the light homogenizer 93 is a light guide rod similar to the light homogenizer 50, and includes an entrance end face 93a, an exit end face 93c, and a reflective interface 93b or peripheral surface. The light homogenizer 93 is different from the light homogenizer 50 in that the reflective interface 93b is tapered from the entrance end face 93a toward the exit end face 93c, or in a shape of a frustum of a cone. Other features of the light homogenizer 93, such as a material, are the same as those of the light homogenizer 50.

The entrance end face 93a can be set large by the form of the frustum of the cone. A plurality of the light source units 31-33 can be directed together to the entrance end face with the large area. Incident light from the light source units 31-33 is diffused in the radial direction during travel in the light homogenizer 93, so that distribution of the light amount can be regularized. The exit end face 93c of the light homogenizer 93 has a diameter equal to that of the light guide device 43. When the endoscope 11 is connected to the light source apparatus 13, the exit end face 93c is opposed to an entrance end face of the light guide device 43. Optical paths from the light source units 31-33 are (aligned and) coupled together as a single path at the exit end face 93c of the light homogenizer 93. The photo sensor S1 is attached to the outside of the reflective interface 93b of the light homogenizer 93.

It is possible by use of the light homogenizer 93 to simplify the construction as the number of the parts is reduced.

In the above embodiments, the light homogenizer is shaped circularly as viewed in a cross section. However, the light homogenizer can be shaped in other forms, such as quadrilateral, pentagonal and hexagonal forms as viewed in a cross section. In the above embodiments, a light guide rod is the light homogenizer. However, a light guide rod of the invention may be a device other than the light homogenizer in which light is reflected internally by total reflection to travel in the optical axis direction.

In the above embodiments, a plurality of light source units or modules are used. However, only one light source unit may be used. In the above embodiments, the light source units 31 with phosphor are combined with the light source units without phosphor. However, it is possible for all light source units to have phosphor. Furthermore, all light source units can be a type without phosphor. It is possible for a light source apparatus to have three light source units for emitting B, G and R light.

In the above embodiments, the light emitting devices of semiconductor are laser diodes. However, light emitting devices can be a light emitting diode (LED), electroluminescence (EL) LED, electroluminescence (EL) element, and the like. The light source may have a structure without light emitting devices of semiconductors, namely may have a lamp such as a xenon lamp and halogen lamp. Those are characterized in a constant light amount and are controlled for exposure control by adjusting an aperture stop device. In use of the light emitting devices, the exposure is controlled by adjusting a drive current in place of use of an adjustable aperture stop device. The exposure control with higher precision is required for the light emitting devices than for a xenon lamp or halogen lamp. The feature of the invention in which the light source is adjusted in output adjustment in a simplified structure is particularly advantageous in the light source including the light emitting devices of semiconductor.

In the above embodiments, images of plural colors are acquired simultaneously. The micro color filters of blue, green and red are used and separate the white light. However, it is possible to use the feature of the invention in a frame sequential imaging, in which a monochromatic imaging unit without color filters is used and acquires color images one after another.

In the above embodiment, the processing apparatus is originally discrete from the light source apparatus. However, the processing apparatus may be combined with the light source apparatus in a single composite apparatus. Also, the endoscope system of the invention may include an ultrasonic endoscope having an ultrasonic transducer in combination with a processing apparatus.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. Alight source apparatus for supplying an endoscope with light, comprising:
at least one light source unit for generating said light;
a transparent light guide rod, including an entrance end face, disposed on a proximal side in an optical axis direction, for receiving entry of said light, an exit end face, disposed on a distal side in said optical axis direction, for exiting said light from said entrance end face, and a reflective interface, formed between said entrance end face and said exit end face, for total reflection of said light in an internal manner and for directing said light in said optical axis direction;
at least one photo sensor, disposed on said reflective interface, for measuring a light amount of said light.

2. Alight source apparatus as defined in claim 1, wherein said light guide rod includes a light homogenizer for regularizing said light amount of said light from said exit end face in a radial direction.

3. Alight source apparatus as defined in claim 1, further comprising an adhesive agent, having a higher refractive index than said light guide rod, for attaching said photo sensor to said reflective interface.

4. A light source apparatus as defined in claim 1, wherein said at least one light source unit is plural light source units;
further comprising an integrating device, disposed upstream of said light guide rod, for aligning optical paths of said light from said plural light source units together and directing said light to said light guide rod.

5. Alight source apparatus as defined in claim 4, further comprising:
a driver for driving said light source units by supplying power;
a lighting control unit, connected with said driver, operated if a change occurs in said light amount measured by said photo sensor, for canceling said change by adjusting said power.

6. A light source apparatus as defined in claim 4, wherein said plural light source units include first and second light source units between which wavelengths of said light are different from one another.

7. A light source apparatus as defined in claim 6, wherein said at least one photo sensor is a plurality of photo sensors.

8. A light source apparatus as defined in claim 6, wherein at least one of said first and second light source units generates special light for special light imaging.

9. A light source apparatus as defined in claim 6, wherein at least one of said first and second light source units includes a light emitting device of semiconductor.

10. A light source apparatus as defined in claim 9, wherein said light emitting device is a laser diode.

11. A light source apparatus as defined in claim 6, wherein said first light source unit includes:
a light emitting device of semiconductor for generating first light;
phosphor for generating fluorescence by excitation with said first light, to emit mixed light by addition of said fluorescence to said first light.

12. A light source apparatus as defined in claim 11, wherein said at least one photo sensor includes:
a first photo sensor sensitive to a first wavelength band of said first light;
a second photo sensor sensitive to a second wavelength band being different from said first wavelength band and containing a wavelength of said fluorescence.

13. A light source apparatus as defined in claim 4, wherein said integrating device includes:
plural input fiber ends, each of which is constituted by a fiber bundle of plural optical fibers, for receiving said light from one of said plural light source units;
an output fiber end for emitting said light to said light guide rod;
a routing section for collecting said optical fibers from said plural input fiber ends into one fiber bundle in said optical axis direction, to constitute said output fiber end.

14. A light source apparatus as defined in claim 4, further comprising an optical coupler, having said light guide rod, and said integrating device formed to extend upstream of said light guide rod.

15. Alight source apparatus as defined in claim 1, further comprising:
a receptacle connector for containing at least a portion of said light guide rod on a side of said exit end face, and for connection with a proximal connector of said endoscope;
a monitoring unit for monitoring reflected light on said reflective interface with said photo sensor upon application of said light from said light source unit through said light guide rod, and for checking whether connection between said receptacle connector and said proximal connector is appropriate by evaluating said reflected light.

16. An endoscope system comprising:
an endoscope; and
a light source apparatus for supplying said endoscope with light;
said light source apparatus comprising:
at least one light source unit for generating said light;
a transparent light guide rod, including an entrance end face, disposed on a proximal side in an optical axis direction, for receiving entry of said light, an exit end face, disposed on a distal side in said optical axis direction, for exiting said light from said entrance end face, and a reflective interface, formed between said entrance end face and said exit end face, for total reflection of said light in an internal manner and for directing said light in said optical axis direction;
at least one photo sensor, disposed on said reflective interface, for measuring a light amount of said light.

17. An endoscope system as defined in claim 16, wherein said light guide rod includes a light homogenizer for regularizing said light amount of said light from said exit end face in a radial direction.

18. An endoscope system as defined in claim 16, further comprising an adhesive agent, having a higher refractive index than said light guide rod, for attaching said photo sensor to said reflective interface.

19. An endoscope system as defined in claim 16, wherein said at least one light source unit is plural light source units;
further comprising an integrating device, disposed upstream of said light guide rod, for aligning optical paths of said light from said plural light source units together and directing said light to said light guide rod.
